(19) United States
(12) Patent Application Publication
(10) Pub. No.: US 2014/0357498 A1
(43) Pub. Date: Dec. 4, 2014

(54) Compositions and Methods for the Detection of DNA Cleavage Complexes

(71) Applicants: Trustees of the University of Pennsylvania, Philadelphia, PA (US); The Children's Hospital of Philadelphia, Philadelphia, PA (US)

(72) Inventors: Carolyn A. Felix, Ardmore, PA (US); Brian A. Gregory, Swedesboro, NJ (US); Li-San Wang, Center Valley, PA (US); Marie L. Carrillo, Philadelphia, PA (US)

(73) Assignee: The Children's Hospital of Philadelphia, Philadelphia, PA (US)

(21) Appl. No.: 14/122,277

(22) PCT Filed: May 29, 2012
(86) PCT No.: PCT/US12/39834

§ 371 Date: Jul. 16, 2014

Related U.S. Application Data
(60) Provisional application No. 61/490,975, filed on May 27, 2011.

(51) Int. Cl. C12Q 1/68; G01N 33/53

(52) U.S. Cl. CPC C12Q 1/6869; G01N 33/5308; C12Q 1/6886; C12Q 2600/16; C12Q 2600/156
USPC 506/2; 435/6.12; 435/6.11; 506/16

(57) ABSTRACT

Compositions, methods, and kits for identifying protein-nucleic acid complexes, particularly DNA topoisomerase II-DNA complexes, are disclosed.

This application claims priority under 35 U.S.C. §119(e) to U.S. Provisional Patent Application No. 61/490,975, filed on May 27, 2011. The foregoing application is incorporated by reference herein.

This invention was made with government support under Grant Nos. CA80175 and CA77683 awarded by the National Institutes of Health. The government has certain rights in the invention.

### FIELD OF THE INVENTION

The present invention relates to the fields of molecular biology and oncology. More specifically, the invention provides compositions and methods for identification of the sequences within protein-nucleic acid complexes, particularly DNA-topoisomerase II complexes with genomic DNA.

### BACKGROUND OF THE INVENTION

Several publications and patent documents are cited throughout the specification in order to describe the state of the art to which this invention pertains. Each of these citations is incorporated herein by reference as though set forth in full.

DNA damage mediated by topoisomerase II poisons has been implicated as a cause of leukemia characterized by balanced chromosomal translocations. Leukemia can be acute or chronic, lymphoid or myeloid, secondary or de novo. Various forms of leukemia are caused by chromosomal translocations, with similar translocations occurring in secondary and de novo cases. Rearrangement of the MLL gene at chromosome band 11q23 is the most common chromosomal translocation in oncogenic cells in infants with leukemia and patients with secondary leukemia after treatment with topoisomerase II poisons (Howlader et al. (2011) SEER Cancer Statistics Review, 1975-2008. National Cancer Institute. Bethesda, Md.; Felix et al. (2006) DNA Repair (Amst), 5:1093-1108).

### SUMMARY OF THE INVENTION

In accordance with the instant invention, methods for identifying sequences bound by a protein-nucleic acid complex are provided. In a particular embodiment, the method comprises enzymatically cleaving the nucleic acid from the protein-nucleic acid complex. In a particular embodiment, the method comprises cleaving a protein-nucleic acid complex (optionally isolated from a cell (e.g., by immunoprecipitation)) by contacting the protein-nucleic acid complexes with a cleaving enzyme (e.g., a phosphatase); contacting the released nucleic acid molecules (optionally purified/isolated) with a polymerase and a polynucleotide kinase (e.g., to fill in overhangs); amplifying the repaired DNA by adaptor PCR; and identifying the sequence of the amplified nucleic acid molecules, thereby identifying the sequences present in the protein-nucleic acid complexes. The cells may be obtained from a subject (e.g., a human). In a particular embodiment, the cells or subject have been exposed to at least one agent being screened for the ability to modulate formation/stabilization of the protein-nucleic acid complexes (e.g., a topoisomerase II poison). In yet another embodiment, the nucleic acid molecules of the protein-nucleic acid complex are fragmented, at least prior to the adaptor PCR.

In accordance with another aspect of the instant invention, kits for practicing the methods of the instant invention are provided.

### BRIEF DESCRIPTION OF THE DRAWINGS

FIG. 1 provides a schematic of topoisomerase II poisons v. inhibitors. Certain agents act as topoisomerase II poisons (FIG. 1A), which initiate DNA damage by increasing the level of steady-state cleavage complexes. This can occur by either increasing the forward rate of cleavage or decreasing the reverse rate of religation. These poisons differ from agents that function as catalytic inhibitors of topoisomerase II (FIG. 1B), which disrupt its enzymatic properties.

FIG. 2 provides a schematic model of the formation of MLL chromosomal translocations in secondary leukemia. Chemotherapy drugs that act as topoisomerase II poisons increase the level of steady-state cleavage complexes. Etoposide, for example, increases the level of steady-state complexes by decreasing the reverse rate of DNA religation. Each subunit of the topoisomerase II homodimer introduces a 4-base staggered nick in the DNA through formation of a phosphodiester linkage. The nick in each strand is stabilized through occupancy of a separate drug molecule. This is known as the two-drug model and can lead to double-strand breaks in the DNA. The DNA repair mechanism of non-homologous end joining (NHEJ) then creates the fusion gene consisting of MLL and a partner gene. This occurs with no or few bases either gained or lost from the native MLL and partner genes during the translocation.

FIG. 3 provides a schematic model for formation of MLL chromosomal translocations in leukemia in infants. In leukemia in infants, there commonly are several hundred base pair duplicated segments of MLL or partner genes at cloned genomic breakpoint junctions. This stems from the presence of single-strand nicks in DNA, which can form in two different ways. The first is the result of a kinetic intermediate of the double-strand breaks caused by topoisomerase II. The second is a result of the two-drug model, which states that each topoisomerase II subunit of the homodimer is independently associated with a molecule of the drug. Therefore, at low drug concentrations, only one subunit may be associated with a molecule of the drug, and a single-strand nick can form. When two separate single-strand nicks occur, a break is created with long 5′ overhangs. This can occur in MLL or a partner gene. The overhangs are resolved through DNA repair mechanisms that involve template-directed polymerization and non-homologous end-joining (NHEJ) and can result in formation of the fusion gene of MLL with a partner gene.

FIG. 4 provides a schematic of the isolation, purification, and quantization of DNA in TOP2A cleavage complexes and TOP2A.

FIG. 5A is a schematic of the MLL breakpoint cluster region (bcr) and primer design for Q-PCR. FIG. 5B provides primer sequences for Q-PCR. Forward primers are SEQ ID NOs: 1-13, from top to bottom, and Reverse primers are SEQ ID NOs: 14-26, from top to bottom. FIG. 5C provides repeats within the MLL bcr.

FIG. 6 provides a schematic of whole genome sequencing to map and quantify DNA ends by TOP2A cleavage genome-wide.

FIG. 7 provides a Western blot analysis of effects of different lysis buffers on TOP2A recovery after immunodepletion of sonicated lysate. Fifty million cells were subjected to three different lysis procedures using either: 1) RIPA Lysis Buffer, 2) CHAPS Lysis Buffer or 3) Cell Membrane Lysis Buffer followed by Nuclear Membrane Lysis Buffer. Ten μL of α-TOP2A mouse IgG1 was added to the sonicated lysate followed by rotation at 4° C. for one hour. Fifty μL of Protein G magnetic beads were added, followed by incubation at room temperature for 10 minutes. Samples were electrophoresed and protein was then transferred to 0.45 micron PVDF filter. The membrane was hybridized with α-TOP2A rabbit IgG or α-ACTB (β-actin) monoclonal mouse IgG1.

FIG. 8 provides a graph of Q-PCR analysis of DNA released from bound fraction after CIP treatment using primers designed around translocation breakpoint hotspots. IP was performed using either 10 μg of α-TOP2A rabbit IgG or 10 mg of α-BECN1 IgG (negative IP control) and incubation with 50 μL of Protein G magnetic beads followed by successive immunodepletion two additional times. DNA was released from cleavage complexes by CIP treatment and purified by phenol-chloroform extraction followed by chloroform extraction and subsequent ethanol precipitation before Q-PCR. Amplification of respective MLL bcr amplicons A-F in DNA released from cleavage complexes after purification is plotted as a percentage of that in input (5% of sonicated lysate). Amplicons A-F are concentrated around translocation breakpoint hotspots in secondary leukemia and leukemia in infants (See FIG. 5). The two arrows represent the location (not drawn to scale) of known secondary and infant translocation breakpoint hotspots. Results below show that the second amplicon, ‘B’, displays the highest degree of amplification in DNA from bound fractions that had incubated with α-TOP2A rabbit IgG.

FIG. 9 provides a graph showing the Q-PCR analysis of DNA released by CIP treatment showing quantitative enrichment of DNA amplicon proximal to the MLL translocation breakpoint hotspot in bound fractions obtained using α-TOP2A antibody for immunodepletion over that obtained using negative control antibody α-BECN1 for immunodepletion in mononuclear cells from three untreated cord blood samples.

FIG. 10 provides images of Western blot analyses showing the immunodepletion of TOP2B from untreated CEM cells.

FIG. 11 provides images of two replicates of a Western blot assay of input, non-bound fraction, and immunoprecipitate (IP) from TOP2A immunoprecipitations of cleavage complexes comprising the 8.3 kb double stranded fragment of the MLL bcr as substrate and native TOP2A enzyme or TOP2A in the presence of the TOP2 poisons etoposide, genistein, or p-benzoquinone.

### DETAILED DESCRIPTION OF THE INVENTION

Leukemia is a form of cancer that begins in the blood forming cells and causes an unregulated hyperproliferation of abnormal white blood cells. According to the most recent SEER data, 244,272 people in the U.S. have been diagnosed with leukemia at one point in their life. In 2010, an estimated 43,050 new cases would be diagnosed and 21,840 people would die of the disease (Howlader et al. (2011). SEER Cancer Statistics Review, 1975-2008. National Cancer Institute. Bethesda, Md.). Leukemia is characterized as one of four major types based on the rate of progression of the disease, as well as, the precursor cell that is affected. These four types are Acute Lymphoblastic Leukemia (ALL), Acute Myeloid Leukemia (AML), Chronic Lymphoblastic Leukemia (CLL) and Chronic Myeloid Leukemia (CML).

Acute leukemia is defined by a rapid progression of the disease, when untreated, and the accumulation of immature, nonfunctional white blood cells in the bone marrow and the blood. This hyperproliferation causes crowding in the bone marrow that prevents the development of normal blood cells, leading to related manifestations in patients with leukemia. More specifically, the insufficient number of megakaryocytes (platelet precursors) and platelets may cause patients to become vulnerable to bruising and bleeding. Anemia is commonly observed because of the lack of erythrocyte precursors in the bone marrow, which leads to fatigue, pallor and shortness of breath. In addition, the patient's immune system may be greatly weakened as a result of the inadequate production of normal leukocytes, increasing susceptibility to infection. Chronic leukemia is defined by a slow progression of the disease, when untreated, and often manifests as an increased number of more mature blood cells. This prolongs the accumulation of abnormal and nonfunctional blood cells. A patient with chronic leukemia may not have any symptoms early in the disease. However, as the number of abnormal blood cells rises, manifestations of the disease will begin to appear.

Acute Lymphoblastic Leukemia, or ALL, is characterized by an accumulation of immature lymphocytes called lymphoblasts, in the bone marrow. The excessive numbers of nonfunctional lymphoblasts prevents the normal formation of other blood cells. The current five-year survival rate of patients with ALL of all ages is 66.4%, according to the most recent SEER data (Howlader et al. (2011). SEER Cancer Statistics Review, 1975-2008. National Cancer Institute. Bethesda, Md.). In addition, ALL is the most prevalent form of pediatric cancer. Acute Myeloid Leukemia, or AML, is characterized by an accumulation of immature myeloid cells called myeloblasts. Symptoms of AML are similar to those of ALL, stemming from overcrowding that causes a lack of normal blood cell production in the bone marrow. The five-year survival rate of patients with AML spanning all ages is 22.6%, according to the most recent SEER data (Howlader et al. (2011). SEER Cancer Statistics Review, 1975-2008. National Cancer Institute. Bethesda, Md.).

One major cause of leukemia is the occurrence of a chromosomal translocation that transforms normal cells into oncogenic cells. Topoisomerase II poisons have been implicated as a cause of chromosomal translocations in certain types of leukemia. Both de novo leukemia and treatment-related (secondary) leukemia associated with previous chemotherapy with topoisomerase II poisons present with similar genetic aberrations. In AML especially, these genetic aberrations are often but not always associated with particular cellular morphologies. This includes the MLL translocation (often FAB M4 or M5 morphology), the t(8;21) translocation (often FAB M2 morphology), the inv(16) (FAB M4 with eosinophilia) and the t(15;17) translocation in APL (FAB M3 morphology), a subset of AML. MLL rearrangements are the most common chromosomal abnormality in ALL in infants and in secondary leukemia after treatment with topoisomerase II poisons (Felix et al. (2006) DNA Repair (Amst) 5:1093-1108).

Mixed Lineage Leukemia, or MLL, is a molecular and cytogenetic subset of ALL, AML and myelodysplastic syndrome, a pre-AML disease. Mixed Lineage Leukemia is characterized by a rearrangement of the MLL gene with a partner gene, of which there are more than 70, resulting in a balanced chromosomal translocation and the production of an oncogenic fusion protein in the leukemia cells (Marschalek, R. (2011) Br. J. Haematol., 152:141-154; Zieminvanderpoel et al. (1991) Proc. Natl. Acad. Sci., 88:10735-10739). Cells with MLL rearrangements can be biphenotypic, expressing surface markers of both myeloid and lymphoid lineages (Al-Seraihy et al. (2009) Haematologica, 94:1682-1690). MLL can also be bilineal, where both myeloid and lymphoid cells are leukemic and have an MLL rearrangement (Derwich et al. (2009) Leuk. Res., 33:1005-1008). In addition, it has been shown that MLL can undergo lineage switch. In one study, a patient treated for MLL-rearranged ALL went into remission and then subsequently developed MLL-rearranged AML (Trikalinos et al. (2009) Br. J. Haematol., 145:262-264).

The MLL gene is located on chromosome 11, band q23. It is important to note that MLL has been evolutionarily conserved, with a high degree of homology to the Drosophila melanogaster trithorax (trx) gene, implicating its critical importance (Djabali et al. (1992) Nat. Genet., 2:113-118; Tkachuk et al. (1992) Cell, 71:691-700). The trx protein is a homeotic gene regulator of the timing, proper development and layout of body structures (Breen et al. (1991) Mech. Dev., 35:113-127). The MLL protein is a global transcriptional regulator, involved in activation or repression of specific target genes by epigenetic mechanisms. MLL's role in epigenetic regulation of transcription is a function of its histone H3 lysine 4 (H3K4)-specific methyltransferase activity. As a histone methyltransferase (HMT), MLL adds a methyl group to the lysine side chain of the H3 histone (Milne et al. (2002) Mol. Cell, 10:1107-1117; Southall et al. (2009) Mol. Cell, 33:181-191). It was previously believed that the main functionality of histones was to ensure proper packing of DNA into organized chromatin. Chromatin consists of nucleosomes which contain DNA wrapped around a histone core made up of two sets of H2A, H₂B, H3 and H4 histones (Luger et al. (1997) Nature, 389:251-260). However, it is now understood that histones also play a key role in gene expression by altering the accessibility of genes to transcription machinery. This function of histones is regulated by post-translational modifications, such as methylation, allowing for cell type-specific gene expression (Wolffe et al. (1999) Nucleic Acids Res., 27:711-720; Rice et al. (2001) Curr. Opin. Cell Biol., 13:263-273). In this way, MLL's function of methylating the lysine residue within the H3 histone is a means of regulating gene expression.

MLL been shown to regulate the expression of HOX genes, most likely by way of its HMT activity (Milne et al. (2002) Mol. Cell, 10:1107-1117). HOX genes are categorized as Class 1 homeobox-containing genes that encode for regulators of transcription during development. M11 heterozygous (+/−) mice have been shown to display altered Hox gene expression and resultant patterning defects in various body structures (Yu et al. (1995) Nature, 378:505-508). MLL also regulates the expression of genes including HNF-3/BF-1, FBJ, and PE31/TALLA-1, which are involved in oncogenic transformation. In addition, MLL targets include tumor suppressor proteins, such as p27kip1 and GAS-1. In mouse models, M11 has been shown to localize with microRNAs that function in blood cell development and leukemia (Guenther et al. (2005) Proc. Natl. Acad. Sci., 102:8603-8608; Scharf et al. (2007) Oncogene, 26:1361-1371; Ansari et al. (2010) Febs Journal, 277:1790-1804).

Fundamental to understanding MLL's targets is the knowledge that MLL is a major determinant of proper hematopoiesis in both the embryonic and adult systems. In murine models, embryonic stem (ES) cells that lack M11 are unable to differentiate into hematopoietic stem cells (HSCs), which results in the obstruction of normal blood cell development. It has been shown that these M11-deficient cells also have a decrease in Hox gene expression, specifically Hoxa7, Hoxa9, Hoxal0 and Hoxa4 (Yagi et al. (1998) Blood, 92:108-117; Hess et al. (1997) Blood, 90:1799-1806). Interestingly, when Hox gene expression is reactivated in M11-deficient cells, differentiation into hematopoietic stem cells takes place. This implicates MLL's regulation of Hox genes as a potential mechanism for its function in HSC production during embryogenesis (Ernst et al. (2004) Developmental Cell, 6:437-443; Ernst et al. (2004) Curr. Biol., 14:2063-2069). In human HSCs, the loss of MLL causes cell-cycle re-entry and differentiation. Therefore, MLL is crucial to maintain the quiescent state of HSCs and their ability to self renew. This indicates that MLL functions in the homeostasis of adult bone marrow by maintaining the stem cell population. MLL is also necessary for the proliferation of the common myeloid progenitor (CMP) and common lymphoid progenitor (CLP) cells (Jude et al. (2007) Cell Stem Cell, 1:324-337).

Returning to MLL's role in leukemia, MLL chromosomal rearrangements cause the formation of an MLL fusion gene that can lead to leukemogenesis. The genomic translocation breakpoints in the MLL gene most frequently occur in a region that spans 8.3 kb between exon 5 and exon 11 of MLL (Rasio et al designation), known as the breakpoint cluster region (bcr) (Rasio et al. (1996) Cancer Res., 56:1766-1769; Thirman et al. (1993) N. Eng. J. Med., 329:909-914; Gu et al. (1994) Cancer Res., 54:2327-2330; Felix et al. (1995) Blood, 85:3250-3256). Within the MLL bcr, there is a 3′ bias to the location of translocation breakpoints (Broeker et al. (1996) Blood, 87:1912-1922). This is true for both leukemia in infants, where maternal-fetal exposure to naturally occurring topoisomerase II poisons takes place, as well as in treatment-related (secondary) leukemia that occurs after chemotherapy with topoisomerase II poisons for a primary cancer. The breakpoints are most often located within an intron, which is also the case for the breakpoint in the partner gene. This allows for the fusion transcript to be in-frame. There are currently 71 partner genes that have been found to fuse with MLL (see, e.g., U.S. patent application Ser. Nos. 11/764,568; 11/199,544; and 12/487,789). However, the most common partner genes are AF4, AF9, ENL, AF10, AF6 and ELL (Marschalek, R. (2011) Br. J. Haematol., 152:141-154; Meyer et al. (2009) Leukemia, 23:1490-1499; Robinson et al. (2009). Specific MLL Partner Genes in Infant Acute Lymphoblastic Leukemia (ALL) Associated with Outcome Are Linked to Age and White Blood Cell Count (WBC) at Diagnosis: A Report on the Children's Oncology Group (COG) P9407 Trial. 51st ASH Annual Meeting and Exposition.).

The fusion gene is considered a deregulated transcriptional regulator. MLL fusion transcripts cause a deregulation of Hox gene expression, which contributes to transformation. Studies in murine models have shown that overexpression of certain Hox genes, specifically HoxA7 and HoxA9, are required for leukemogenesis in many MLL leukemias. The products of these Hox genes may cause an increase in the self-renewal capacity of hematopoietic progenitors and a decrease in differentiation (Ayton et al. (2003) Genes Dev., 17:2298-2307; Armstrong et al. (2002) Nat. Genet., 30:41-47; Rozovskaia et al. (2001) Oncogene, 20:874-878). Other studies in murine models have shown that HoxA7 and HoxA9 may regulate downstream effects of leukemogenesis and determine the rate of onset of MLL (Ayton et al. (2001) Oncogene, 20:5695-5707; Yokoyama et al. (2004) Mol. Cell. Biol., 24:5639-5649).

Light was first shed on the cause of MLL rearrangements when a new class of chemotherapy agents was brought to the clinic and caused a rise in secondary leukemia with balanced chromosomal translocations. These agents functioned as poisons of topoisomerase II by turning this enzyme into a cellular toxin. Resultantly, researchers began to investigate the connection between topoisomerases and leukemia (Deweese et al. (2008) Nucleic Acid Res., 36:4883-4893).

Vital to understanding the relationship between topoisomerase II poisons and the development of MLL translocations is knowledge of the normal cellular functions of topoisomerase. When replication or transcription machinery moves along DNA and separates complementary DNA strands, supercoils are created. In order to release the tension in DNA caused by supercoils, topoisomerases create transient breaks in the double helix. This allows the DNA to unwind and return to its relaxed state. In other words, topoisomerases control the topological state of DNA by regulating over-winding and under-winding, as well as removing knots and tangles that may form. Topoisomerases are ubiquitous in the nucleus because of their highly significant role in changing DNA topology from the supercoiled to the relaxed state (Liu et al. (1983) J. Biol. Chem., 258:5365-5370; Liu et al. (1987) Proc. Natl. Acad. Sci., 84:7024-7027; Wang, J. C. (1996) Annu. Rev. Biochem., 65:635-692).

There are two major classifications of topoisomerases. Type I topoisomerases cut one strand of DNA, which unravels around its complementary strand and is subsequently religated. Type II topoisomerases induce a double-strand break, allowing an intact DNA helix to pass through the opening, followed by the religation of both strands. This relieves tension with only a transient change in the structural integrity of DNA. In humans, Type II topoisomerases are subdivided into topoisomerase IIα (TOP2A) and topoisomerase II13 (TOP2B) isoforms (Wang, J. C. (1996) Annu. Rev. Biochem., 65:635-692). The two isoforms are encoded on different genes, yet they display approximately 70% sequence homology. The human topoisomerase IIα gene is located on chromosome 17, band q21-22 and encodes for a 170 kDa protein, while the human topoisomerase IIβ gene is found on chromosome 3, band p24 and encodes for a 180 kDa protein. The two isoforms have different functional roles in the cell. The topoisomerase IIα isoform is necessary for chromosomal segregation during mitosis, as well as for DNA replication. In addition, its levels are regulated throughout the cell cycle, peaking at the G2/M phase. Topoisomerase IIβ is believed to release tension during DNA transcription and its levels are independent of growth status and cell cycle stage. Unlike topoisomerase IIα, the IIβ isoform is not an essential enzyme (Deweese et al. (2008) Nucleic Acid Res., 36:4883-4893; Felix et al. (2006) DNA Repair, 5:1093-1108).

Topoisomerase II is a homodimer and each subunit contains an active site tyrosine residue that cuts one DNA strand. The mechanism of scission involves the breaking of a phosphodiester bond in the DNA and the formation of a covalent phosphodiester bond between the tyrosyl residue on the enzyme and the induced 5′ phosphate residue at the 3′ side of cleavage on the DNA strand (Wang, J. C. (2002) Mol. Cell. Biol., 3:430-440). This also creates a 3′ hydroxyl group on the other side of the break. The DNA-topoisomerase II complex is referred to as the cleavage complex and is normally a fleeting transient intermediate in the enzymatic cycle of the enzyme (Felix et al. (2006) DNA Repair, 5:1093-1108; McClendon et al. (2007) Mut. Res., 623:83-97). Since both subunits of the homodimer form this complex, two separate covalent phosphodiester bonds are created. These two covalent bonds formed from one homodimer are located on opposing strands, four bases apart. Therefore, a four-base 5′ overhang is created when both strands of the DNA are cleaved. It is important to note that because of the enzymatic cycle of topoisomerase IIα, there are times when only one subunit of the homodimer is associated with DNA (Felix et al. (2006) DNA Repair (Amst), 5:1093-1108). In order to religate the break, the 3′ hydroxyl group on DNA attacks the phosphorus in the cleavage complex and reestablishes the phosphodiester bond of the DNA backbone (Liu et al. (1983) J. Biol. Chem., 258:5365-5370; Wang, J. C. (2002) Mol. Cell. Biol., 3:430-440; Zechiedrich et al. (1989) Biochemistry, 28:6229-6236). Topoisomerase II's catalytic activity requires ATP hydrolysis for the intact DNA double helix to pass through the break (Wang, J. C. (2002) Mol. Cell. Biol., 3:430-440). In addition, divalent metal ions are required and may function in a two-metal-ion mechanism. In this way, one of the ions may stabilize the 3′ oxygen on DNA, increasing the rate of formation of the cleavage complex (Deweese et al. (2008) Nucleic Acid Res., 36:4883-4893). As a result of the catalytic cycle of topoisomerase II, these cleavage complexes are normally present at low-steady state levels and are not harmful to the cell (Burden et al. (1998) Biochim. Biophys. Acta, 1400:139-154).

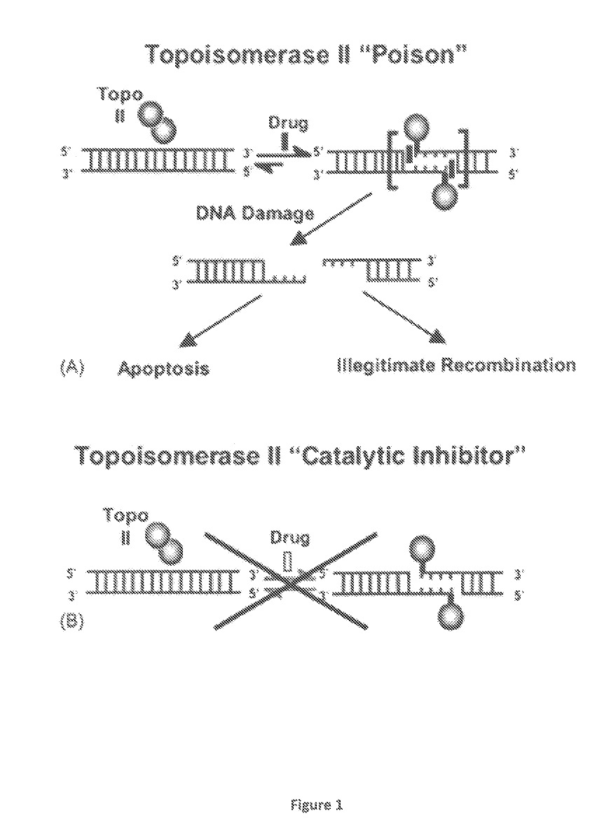

As FIG. 1 shows, there are certain molecules that act as topoisomerase II poisons, while other molecules function as catalytic inhibitors. Topoisomerase II poisons initiate DNA damage by increasing the number of steady-state cleavage complexes. This is done by either increasing the forward rate of cleavage or decreasing the reverse rate of religation. Conversely, catalytic inhibitors act by blocking function along various steps of topoisomerase II's catalytic cycle (Capranico et al. (1997) Cancer Chemother. Biol. Response Modif., 17:114-131; Capranico et al. (1998) Biochim. Biophys. Acta, 1400:185-194). This includes topoisomerase II binding, DNA cleavage, DNA religation or dissociation of the enzyme. When topoisomerase II poisons are present, each of the four-base staggered nicks created by a subunit of the topoisomerase II homodimer (as described above) is stabilized by the occupancy of a separate molecule of the poison. This is known as the two-drug model (Bromberg et al. (2003) J. Biol. Chem., 278:7406-7412). DNA tracking systems, such as replication forks or transcription complexes, which normally move along DNA will reach the cleavage complex and the collision that ensues creates a permanent double-strand DNA break (Felix et al. (2006) DNA Repair, 5:1093-1108; McClendon et al. (2005) J. Biol. Chem., 280:39337-39345). The property of chemotherapy drugs that cause topoisomerase II poisons to damage DNA by increasing cleavage complexes is the reason these drugs are useful in treating cancer. Examples of common topoisomerase II poisons that have been employed as chemotherapeutics and also cause leukemia are the anthracyclines, such as doxorubicin and daunorubicin, and the epipodophyllotoxins, such as etoposide and teniposide. It is important to note that the utilization of teniposide was halted when it was recognized to be associated with very high leukemia risk (Pui, C. H. (1990) Lancet, 336:1130-1131). Another example is mitoxantrone, which is particularly associated with a high risk of developing secondary APL compared to other chemotherapeutics (Mistry et al. (2005) N. Eng. J. Med., 352:1529-1538). Since oncogenic cells exhibit hyperproliferation, they also have higher quantities of topoisomerase II for use during replication. As a result, oncogenic cells are more vulnerable to the effects of topoisomerase II poisons compared to cells undergoing normal levels of proliferation (Burden et al. (1996) J. Biol. Chem., 271:29238-29244). If the increase in double-strand breaks within the cell meets a certain threshold, activation of apoptosis will occur. In terms of chemotherapy, this would successfully decrease the number of oncogenic cells.

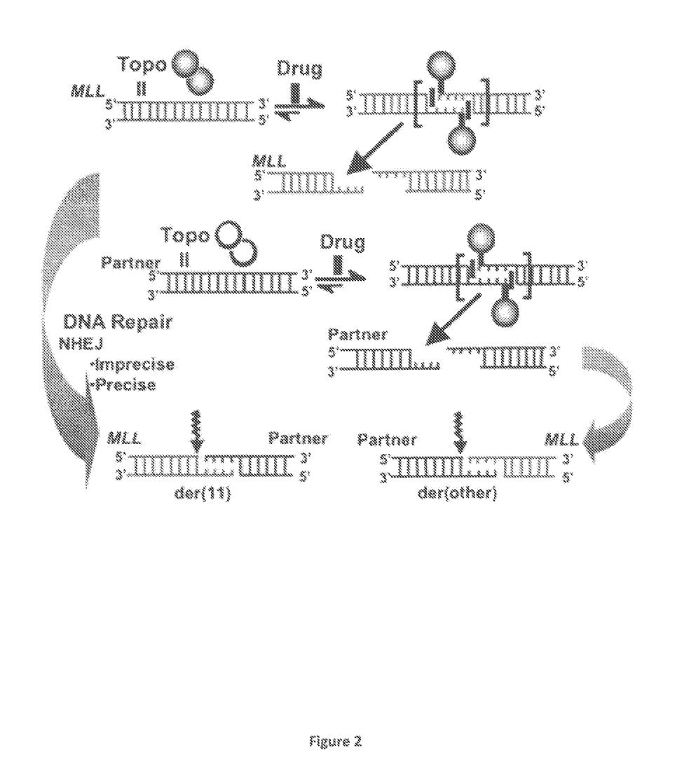

Unfortunately, in some cases (about 2-3%), utilization of topoisomerase II poisons as chemotherapeutics is associated with the devastating treatment complication of leukemia as a secondary cancer (Smith et al. (2010) J. Clin. Oncol., 28:2625-2634). It has been hypothesized that rather than activating apoptosis, the double-strand breaks induced by the poison lead to a balanced chromosomal translocation with a partner gene, which had also undergone a double-strand break (Lovett et al. (2001) Biochemistry, 40:1159-1170; Felix, C. A. (1998) Biochim. Biophys. Acta, 1400:233-255). Further, it also has been hypothesized that this occurs by way of the DNA repair mechanism of non-homologous end joining (NHEJ) which creates an oncogenic fusion gene. Usually, in the treatment-related cases, molecular cloning of the reciprocal genomic translocation breakpoint junctions has shown that no or few bases are either gained or lost from the native MLL and partner genes during the translocation (See FIG. 2) (Felix et al. (2006) DNA Repair, 5:1093-1108; Whitmarsh et al. (2003) Oncogene, 22:8448-8459; Lovett et al. (2001) Proc. Natl. Acad. Sci., 98:9802-9807). There is also a specific genotype, CYP3A4-W, which increases the risk of developing treatment-related leukemia. Cytochrome P-450 (CYP) 3A4 metabolizes epipodophyllotoxins and creates quinone metabolites that are damaging to DNA by functioning as topoisomerase II poisons (Lovett et al. (2001) Biochemistry, 40:1159-1170). Therefore having the wildtype CYP3A4-W genotype increases a patient's risk of developing secondary leukemia. Alternatively, having the variant genotype, CYP3A4-V, may decrease the metabolism of epipodophyllotoxins, therefore, lowering the risk of secondary leukemia (Lovett et al. (2001) Biochemistry, 40:1159-1170; Felix et al. (1998) Proc. Natl. Acad. Sci., 95:13176-13181).

The actions of topoisomerase II poisons are not specific to one isoform of the enzyme. The precise function of each isoform in the pathway to oncogenesis is unclear. Regardless, there is also evidence that the β isoform may play a major role in the development of leukemia after treatment with topoisomerase II poisons (Azarova et al. (2007) Proc. Natl. Acad. Sci., 104:11014-11019; Azarova et al. (2010) Biochem. Biophys. Res. Commun., 399:66-71).

Topoisomerase II poisons are also implicated in causing leukemia in infants. Twin studies have been used as a means to demonstrate that the originating MLL translocation occurs in utero and is passed through placental anastomoses to the other twin (Ford et al. (1993) Nature, 363:358-360; Greaves et al. (2003) Blood, 102:2321-2333.). When analyzing infant twins under one year of age with MLL-rearranged leukemia, there is almost 100% concordance of the disease. Blood samples from twin studies have confirmed that twins share the same MLL chromosomal translocation. In order for the exact abnormality to be shared between twins, the de novo mutation must have occurred in utero, with blood mixing as a means of transferring cells with the translocation from one twin to the other (Ford et al. (1993) Nature, 363:358-360; Super et al. (1994) Blood, 83:641-644. Analysis of neonatal blood spots on Guthrie cards and detection of the MLL translocation in non-twin cases has confirmed this hypothesis (Gale et al. (1997) Proc. Natl. Acad. Sci., 94:13950-13954.).

It has been shown that approximately 75% of all infant patients with leukemia have an MLL-rearrangement in their leukemic cells (Cimino et al. (1993) Blood, 82:544-546; Pui et al. (1990) Blood, 76:1449-1463). According to the COG P9407 Trial, infants under one year of age with MLL-rearranged ALL have a five year event-free survival rate of 38.8% compared to infant patients with ALL with germline (non-rearranged) MLL who have a 66.2% event-free survival rate. Although MLL-rearranged AML in infants is also common, event-free survival rates are not significantly different from AML in infants where MLL is not rearranged (Cimino et al. (1993) Blood, 82:544-546; Pui, C. H. (1996) Curr. Opin. Hematol., 3:249-258; Satake et al. (1999) Leukemia, 13:1013-1017). The low survival rate of infants with leukemia is related to the prevalence of poor prognostic factors including young age, higher white blood cell count and the occurrence of the MLL translocation. In addition, infants experience a higher rate of relapse due to resistance to chemotherapy regimens, as well as, toxicities to which infants are uniquely vulnerable (Pui et al. (2000) Leukemia, 14:684-687; Hilden et al. (2006) Blood, 108:441-451).

The subsequent challenge of understanding MLL rearrangements in infants is determining the cause of the initial molecular genetic lesion. It is believed that the fetus may be exposed to certain substances in utero through maternal circulation that can cause the resulting chromosomal abnormality (Ross et al. (1994) J. Natl. Cancer Inst., 86:1678-1680). Since there was a known relationship between topoisomerase II poisons and secondary leukemia, which also shared a high prevalence of the MLL translocation, researchers wondered if similar poisons could also be implicated in MLL in infants. There are natural molecules found in dietary sources that can act as topoisomerase II poisons including genistein, a substance found in soybeans and coffee, and bioflavonoids, which can be found in fruits, vegetables, dark chocolate and red wine (Felix et al. (2006) DNA Repair (Amst), 5:1093-1108; Spector et al. (2005) Cancer Epidemiology Biomarkers & Prevention, 14:651-655; Yamashita et al. (1990) Biochem Pharmacol., 39:737-744; Ross et al. (2002) Annu. Rev. Nutr., 22:19-34). As a result, the Children's Oncology Group performed a retrospective study utilizing a food frequency questionnaire to determine whether there was a correlation between a mother's consumption of dietary topoisomerase II interacting compounds during pregnancy to the development of leukemia in infants. Results showed that high levels of consumption of foods containing topoisomerase II interacting compounds was associated with an increased risk of MLL-rearranged AML in infants (Ross et al. (1996) Cancer Causes Control, 7:581-590; Spector et al. (2005) Cancer Epidemiol Biomarkers Prey, 14:651-655).

Since foods containing topoisomerase II interacting compounds are relatively common in maternal diets, there seems to be an underlying genetic predisposition that would cause only certain infants to develop an MLL translocation. One factor may involve the variability in which mothers and/or fetuses can process toxins. For example, there have been two studies showing that the risk of developing MLL-rearranged ALL (especially with the AF4 partner gene) increases when there is a decrease in the activity of the NQO1 (NAD(P)H:Quinone Oxidoreductase-1) protein due to a polymorphism in the NQO1 gene. The NQO 1 gene product detoxifies p-benzoquinone, which is a major metabolite of benzene and a topoisomerase II poison (Lindsey et al. (2004) Biochemistry, 43:7563-7574; Lindsey et al. (2005) Chem. Biol. Interact, 153-154:197-205). Therefore, the decreased ability to detoxify topoisomerase II poisons may increase susceptibility to MLL rearrangements (Wiemels et al. (1999) Cancer Res, 59:4095-4099; Smith et al. (2002) Blood, 100:4590-4593).

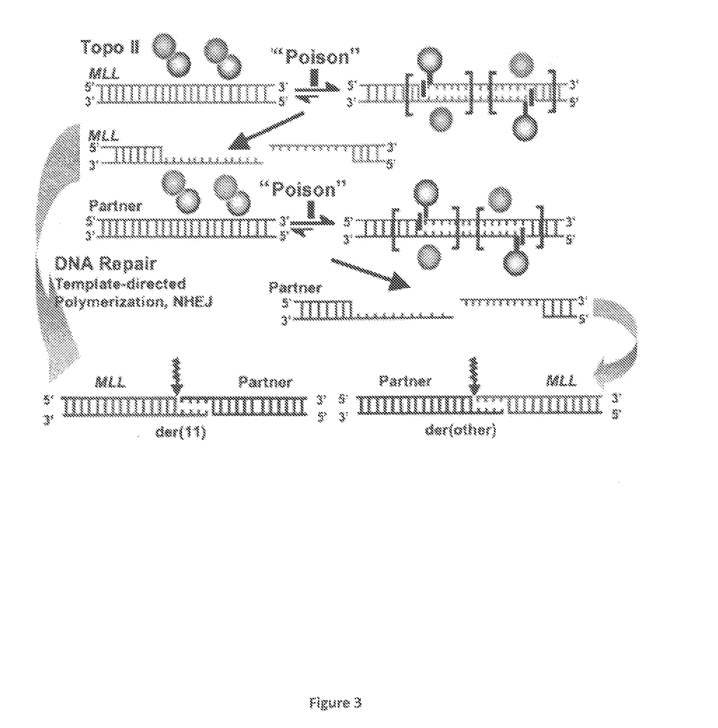

Unlike secondary leukemia, where no or few bases are either gained or lost from the native MLL and partner genes during the translocation, leukemia in infants commonly exhibits several hundred base pair duplicated segments of MLL and/or a partner gene at breakpoint junctions. In the hypothesis in which topoisomerase II DNA damage causes MLL translocations, this stems from the ability of topoisomerase II cleavage to cause single-strand nicks in DNA, as opposed to four-base staggered nicks on both strands of DNA. There are two ways in which single-strand nicks can form. As mentioned above, during the catalytic cycle of topoisomerase II, there are kinetic intermediates in which only one of the homodimer subunits is complexed with DNA, allowing for the formation of a single-strand nick. The second way stems from implications of the double-occupancy, or two-drug model. If there is a low concentration of topoisomerase II poisons present in the cell, only one subunit of the topoisomerase II homodimer may be associated with the poison and, therefore, only one DNA-topoisomerase II phosphodiester linkage will be stabilized by a molecule of the poison. In either scenario, when two independent single-strand nicks are present, a permanent break could form with long 5′ overhangs. This can take place in MLL or a partner gene. Resolution of overhangs through template-directed polymerization and NHEJ could lead to the formation of a transforming fusion gene consisting of MLL and a partner gene (See FIG. 3) (Felix et al. (2006) DNA Repair, 5:1093-1108; Gillert et al. (1999) Oncogene, 18:4663-4671).

Topoisomerase II poisons stabilize cleavage complexes leading to the formation of double-strand DNA breaks that are resolved to create transforming fusion genes. In order to determine the role of topoisomerase II in leukemogenesis, many studies have been performed to help define the relationship between topoisomerase II cleavage sites and translocation breakpoints. Molecular cloning using panhandle PCR as well as other molecular cloning methods have provided evidence of a translocation breakpoint hotspot region 3′ in the MLL bcr. In terms of secondary leukemias, this region spans the bases 6587-6600 (GenBank Accession #: U04737) (Megonigal et al. (2000) Proc. Natl. Acad. Sci., 97:2814-2819; Langer et al. (2003) Genes Chromosomes Cancer, 36:393-401; Megonigal et al. (1997) Proc. Natl. Acad. Sci., 94:11583-11588; Domer et al. (1995) Leukemia, 9:1305-1312). In addition, a breakpoint hotspot region in leukemia in infants has been identified to span the bases 6576-6790 (GenBank Accession #: U04737) (Gillert et al. (1999) Oncogene, 18:4663-4671; Langer et al. (2003) Genes Chromosomes Cancer, 36:393-401; Leis et al. (1998) Leukemia, 12:758-763; Raffini et al. (2002) Proc. Natl. Acad. Sci., 99:4568-4573). In vitro cleavage assays have further defined the specific sites of topoisomerase II cleavage complexes in relation to MLL translocations breakpoints. These assays provide the sequence-specific location of topoisomerase II cleavage sites, which can then be compared to the location of known translocation breakpoints, including those in the translocation breakpoint hotspots 3′ in the MLL bcr (Lovett et al. (2001) Biochemistry, 40:1159-1170; Whitmarsh et al. (2003) Oncogene, 22:8448-8459; Lovett et al. (2001) Proc. Natl. Acad. Sci., 98:9802-9807; Lindsey et al. (2004) Biochemistry, 43:7563-7574; Kolaris et al. (2005). DNA Topoisomerase II Poisons and the Etiology of Acute Leukemia in Infants. 51st ASH Annual Meeting and Exposition; Robinson et al. (2008) Blood, 111:3802-3812). An especially strong topoisomerase II cleavage site, both native and poison-induced (i.e. treatment with p-benzoquinone, genistein, genistin, quercitin or catechin), has been identified at base 6760 in the MLL bcr (Kolaris et al. (2005). DNA Topoisomerase II Poisons and the Etiology of Acute Leukemia in Infants. 51st ASH Annual Meeting and Exposition.). This falls directly within the known translocation breakpoint hotspot region in leukemia in infants. Therefore, these assays have confirmed that there are often one or more topoisomerase II cleavage sites near translocation breakpoints in MLL and partner genes as observed in an in vitro model.

It is also important to note that certain topoisomerase II poisons exhibit sequence selectivity in terms of the location of where they specifically stabilize cleavage complexes along DNA. Etoposide has been shown to cause cleavage where there is a 5′ cytosine residue, or C(−1), directly adjacent to the cleavage site (Lovett et al. (2001) Biochemistry, 40:1159-1170). Conversely, doxorubicin, stabilizes cleavage complexes where there is a 5′ adenine residue, of A(−1) (Capranico et al. (1990) Nucleic Acids Res., 18:6611-6619). Benzoquinone, on the other hand, exhibits a preference for cleavage sites adjacent to a 5′ guanine, or G(−1) (Lindsey et al. (2004) Biochemistry, 43:7563-7574).

An in vitro topoisomerase II cleavage assay has been used extensively in the past to quantify and map the location of cleavage complexes in naked singly 5′ end labeled DNA substrates (plasmid subclones or oligonucleotides) that were treated with topoisomerase II poisons and recombinant topoisomerase II by employing a DNA sequencing ladder primed at the same 5′ end and denaturing polyacrylamide gel electrophoresis (Mistry et al. (2005) N. Engl. J. Med., 352:1529-1538; Lovett et al. (2001) Biochem., 40:1159-1170; Whitmarsh et al. (2003) Oncogene, 22:8448-8459; Lovett et al. (2001) Proc. Natl. Acad. Sci., 98:9802-9807; Lindsey et al. (2004) Biochem., 43:7563-7574; Kolaris et al. (2005) DNA Topoisomerase II Poisons and the Etiology of Acute Leukemia in Infants. 51st ASH Annual Meeting and Exposition; Robinson et al. (2008) Blood, 111:3802-3812). This method has the limitation that the cleavage is being studied outside the nuclear chromatin context of the living cell, even though it has established that there are TOP2A cleavage sites at or near leukemia-associated translocation breakpoints.

The ICE (in vivo complex of enzyme) bioassay also provided methodology to isolate and measure the levels of TOP2A-DNA cleavage complexes from cells treated with various topoisomerase II poisons (Subramanian et al. (2001) Methods Mol. Biol., 95:137-147; Whitmarsh et al. (2003) Oncogene, 22:8448-8459). This assay entails cell lysis followed by ultracentrifugation in CsCl to isolate protein bound DNA and subsequent immunoblotting with an α-TOP2A antibody to detect cleavage complexes. However, the ICE bioassay falls short in that it does not provide any means of determining the locations of the cleavage complexes in the DNA.

Various older methods of isolating DNA in complexes with topoisomerases in a cellular context for subsequent mapping altered the intrinsic state of the cleavage complexes. For example, previous studies in Saccharomyces cerevisiae, murine and human cells employed crosslinking with formaldehyde to artificially stabilize the attachment between topoisomerase and DNA (Kantidze et al. (2006) J. Cell Physiol., 207:660-667; Bermejo et al. (2009) Methods Mol. Biol., 582:103-118; Baldwin et al. (2009) Methods Mol. Biol., 582:119-130; Lyu et al. (2006) Mol. Cell. Biol., 26:7929-7941). Although crosslinking prevents dissociation of the protein from the DNA, the approach can introduce an unnatural association between topoisomerase and DNA and, therefore, a potential for false positive results.

As opposed to artificially stabilizing the cleavage complex, the assays of the instant invention capture protein-nucleic acid complexes (e.g., TOP2A-DNA complexes) in their intrinsic state by taking advantage of the natural covalent phosphodiester bond formed in vivo in cells between TOP2A and DNA. The assay takes on an entirely novel approach by enzymatically removing the covalently attached TOP2A from the DNA with CIP so that the DNA ends can subsequently be precisely mapped (e.g., by high-throughput sequencing). Following IP, the use of CIP was successfully implemented to cleave the covalent bond between DNA and TOP2A, leaving behind an OH residue that could not be religated. This isolated DNA that was previously TOP2A-bound and that is released from TOP2A in its cleaved state can now be used for genome-wide identification of cleavage sites in a cellular context.

In contrast to the precise mapping of DNA double strand breaks from TOP2A cleavage that the method of the instant invention allows, previous strategies to localize topoisomerase and topoisomerase-related proteins (such as Saccharomyces cerevisiae Spoil) covalently bound to DNA, used non-specific nuclease digestion to fragment the DNA and DNA microarray analysis (Bermejo et al. (2009) Methods Mol. Biol., 582:103-118; Gerton et al. (2000) Proc. Natl. Acad. Sci., 97:11383-11390; Reymann et al. (2008) BMC Genomics, 9:324). This approach does not provide the sequence-specific location of where proteins were complexed with DNA. Subsequent microarray analysis used genome-wide probes to obtain signal intensities that identify genomic locations of topoisomerase-DNA complex hotspots and coldspots. This establishes a general layout of complexes throughout the genome, however, it does not provide the sequence-specific location of cleavage complexes. In addition, it is limited to the genomic locations covered by oligonucleotide probes and only at a resolution limited to the sizes of the digested fragments.

Thus, the assay presented here comprises a method of analysis that allows for the identification of the exact sites of DNA double strand breaks created by covalent modification of the DNA in the form of cleavage complexes using sequencing of the sonicated DNA fragments bound to TOP2A that were released from the TOP2A by CIP treatment. This highly novel strategy allows for the first time the determination of precise locations and distribution of cleavage complexes in cells genome wide and the exact sequence composition of genomic regions where cleavage complexes are formed. In addition, it allows for the determination of how sequence selectivity varies in a cellular context in the presence of different poisons and how sites of translocation breakpoints in leukemia in infants and patients with secondary leukemia are related to TOP2A-cleavage complex locations in cells. It also allows for similar correlative analyses of cleavage sites to translocation breakpoints in various different genes when TOP2 poisons may be the etiology of de novo leukemia, particularly in older individuals.

It has been previously determined that there is a bias in MLL translocation breakpoint distribution specifically within the 3′ end of MLL bcr intron 8 (Intron 11 in Nilson numbering system). Molecular cloning of genomic translocation breakpoints in leukemia in patients identified a number of different translocation breakpoint sites in secondary leukemia and leukemia in infants in this region. Even though in vitro topoisomerase II cleavage assays have revealed TOP2A cleavage sites at and near translocation breakpoints at these hotspots as well as at and near translocation breakpoints in other regions of the MLL bcr, it has yet to be determined whether the location of TOP2A-cleavage complexes correlates with translocation breakpoints in a cellular context. Beyond MLL, the instant assay may be used to determine other translocation spots including whether an analogous translocation breakpoint hotspot that was identified in the PML gene in APL with the t(15;17) translocation, which also correlates with an in vitro TOP2A cleavage site, will prove to be a TOP2A cleavage site in a hematopoietic cellular context.

The assays of then instant invention provide quantitative and qualitative comparisons of native to poison-induced topoisomerase II cleavage complexes in a cellular context. MLL rearrangements are the most common genetic abnormality in both infants with leukemia and patients with secondary leukemia after treatment with chemotherapies that act as topoisomerase II poisons. The poor prognosis of patients with MLL-rearrangements and its prevalence among certain leukemia patient populations mandates a better understanding of the causes of the translocations underlying this disease. The highly novel methodology developed, refined and optimized herein provide answers to fundamental questions regarding this model of DNA damage from TOP2A, TOP2B, and from other topoisomerase related enzymes. The instant methods will identify certain regions of the genome which comprise hotspots that are more vulnerable to topoisomerase II-DNA damage induced by topoisomerase II poisons that can form translocations (in which case there would be a biased distribution of topoisomerase II cleavage complexes). The methods will also address whether topoisomerase II cleavage occurs and consequent translocations form without bias genome wide but that translocations in certain areas of the genome provide a selective advantage for leukemia to proliferate. In addition, sequencing (e.g., high-throughput sequencing) will define the exact relationship between topoisomerase II cleavage and MLL translocation breakpoints at single base resolution for the first time ever in a cellular context. Furthermore, the comparison of native and poison-induced cleavage complexes will help elucidate the DNA damaging effects of topoisomerase II poisons as toxins in a cellular context. The above will be of great clinical significance. For example, the instant assay may be used to find tractable markers for screening to follow patients for the development of translocations and an increased risk for leukemia. Besides chemotherapy and dietary TOP2 poisons, similar damage can arise from, e.g. the major benzene metabolite benzoquinone which is found in cigarette smoke and wood smoke and many other sources.

In accordance with the instant invention, methods for identifying sequences present in protein-nucleic acid complexes are provided. The protein-nucleic acid complexes may be in vitro or in a cell. In a particular embodiment, the protein-nucleic acid complexes are in a cell. Cells used in the methods of the instant invention may be obtained/isolated from a subject or may be a cell line. In a particular embodiment, the cell is a cell line used in an experimental model or a bone marrow or blood cell, particularly a hematopoietic stem cell (e.g., CD34⁺ pluripotent hematopoietic stem cells). In a particular embodiment, the cell is a non-hematopoietic cell (e.g., one damaged by chemotherapy or a different genotoxin by a TOP2 damage mechanism).

In a particular embodiment, the protein-nucleic acid complexes of the instant invention are covalently linked complexes that can be enzymatically cleaved. In a particular embodiment, the linkage is a phosphodiester linkage. In a particular embodiment, the covalent linkage is cleavable by a phosphatase. Examples of proteins which form covalent linkages with nucleic acids include, without limitation, topoisomerases, methylases, glycosylases, and RNA-modifying enzymes (see, e.g., Chervin et al. (2007) Methods Enzymol., 425:121-137). In a particular embodiment, the protein is topoisomerase II. The nucleic acid molecule of the protein-nucleic acid complex may be RNA or DNA. In a particular embodiment, the nucleic acid is genomic DNA.

In a particular embodiment of the instant invention, the method comprises cleaving the protein-nucleic acid complexes (e.g., isolated from a cell) by contacting the protein-nucleic acid complexes with a cleaving enzyme (e.g., a phosphatase); contacting the free nucleic acid molecules with a polymerase (e.g. a polymerase which can fill in overhangs) and a polynucleotide kinase, binding adaptors to the free nucleic acid molecules; and identifying the sequence of the nucleic acid molecules (optionally amplified prior to identification of the sequence), thereby identifying the sequences present in the protein-nucleic acid complexes.

In a particular embodiment of the instant invention, the methods comprise exposing the protein-nucleic acid complexes (e.g., exposing the cells or subject) to an agent which modulates formation of protein-nucleic acid complexes. Alternatively, the methods of the instant invention may be used to determine whether an agent (e.g., a polypeptide, protein, nucleic acid molecule, organic compound, small molecule, etc.) modulates formation of protein-nucleic acid complexes. In a particular embodiment, the cells (or subject) have been exposed to a topoisomerase II poison. Topoisomerase II poisons include, without limitation, anthracyclines (e.g., doxorubicin, idarubicin, and daunorubicin), epipodophyllotoxins (e.g., etoposide (and metabolites thereof (e.g., etoposide quinone and etoposide catechol)) and teniposide), aminoacridines (e.g., amsacrine), benzene and benzene metabolites (e.g., benzoquinone, 1,4-benzoquinone), m-AMSA, NK314, XK469, actinomycines (e.g., dactinomycin), and anthracenediones (e.g., mitoxantrone). Other examples of topoisomerase II poisons include, without limitation, dietary TOP2 interacting substances, particularly those to which the fetus can be exposed via the maternal diet. These include but are not limited to substances containing genistein, quercitin, catechin, and various bioflavinoids. In a particular embodiment, the topoisomerase II poison is a chemotherapeutic agent. In yet another embodiment, the protein-nucleic acid complexes are exposed to an environmental factor (e.g., a substance present in the environment; such as benzoquinone), pollutant, or a pesticide.

In a particular embodiment, the protein-nucleic acid complexes are isolated prior to cleavage. The isolation may comprise lysing the cells (optionally in the presence of a protease inhibitor). The isolation step may also comprise the fragmenting of the nucleic acid. In a particular embodiment, the nucleic acids are fragmented to about 100 to about 1000 basepairs in length, particularly to about 500 basepair or less in length. The nucleic acid may be fragmented by any method known in the art including, without limitation, sonication and restriction enzyme digestion. In a particular embodiment, the nucleic acids are sonicated. The protein-nucleic acid complexes may be isolated (e.g., from the cellular lysates) by immunoprecipitation (e.g., with an antibody immunologically specific for the protein). In a particular embodiment, the immunoprecipitation comprises the use of magnetic beads (e.g., protein G magnetic beads). The methods may comprise multiple rounds of immunoprecipitation and/or freezing of the lysates.

As stated hereinabove, the protein-nucleic acid complexes are cleaved by a cleaving enzyme. In a particular embodiment, the cleaving enzyme is a phosphatase such as an alkaline phosphatase. In a particular embodiment, the cleaving enzyme is calf intestinal phosphatase. The free nucleic acids may be subsequently purified (e.g., from the cleaved proteins) by, e.g., immunoprecipitation of the protein. Multiple rounds of immunoprecipitation may be performed.

The free nucleic acids are subjected to repair (e.g., blunting) of the nucleic acid ends. If overhangs are present, the free nucleic acid molecules are contacted with a polymerase to fill in the overhangs. Further, if a phosphatase is used, or another enzyme which eliminates the terminal phosphate of the nucleic acids, then the nucleic acids are contacted with a polynucleotide kinase (e.g., the T4 polynucleotide kinase) to add a phosphate to the nucleic acids.

The repaired nucleic acids are subsequently amplified by adaptor PCR. In a particular embodiment, an overhang (e.g., a 3′ overhang) is added to the repaired nucleic acids. In a particular embodiment, the overhang is a single nucleotide (e.g., an adenosine). The overhangs may be added by a polymerase such as a Taq polymerase or a modified Klenow DNA polymerase. Adaptors may be added to the 5′ and/or 3′ ends of the nucleic acid molecules to be amplified. The adaptors (comprising a known sequence) enable amplification of the nucleic acid molecules. The adaptors are typically short oligonucleotides (e.g., less than about 100 nucleotides, but large enough to be bound by a primer) that may be synthesized by conventional means. The adaptors may be attached by ligation. The adaptors may attach via the overhang. Two different adaptor sequences may be attached to a nucleic acid molecule to be amplified such that one adaptor is attached at one end of the nucleic acid molecule and another adaptor is attached at the other end of the nucleic acid molecule. The free nucleic acids of the instant invention may then be amplified by PCR using adaptor specific primers.

The sequence of the nucleic acids of the instant invention may be determined by any method known in the art. In a particular embodiment, the sequence of the nucleic acid is determined by sequencing of the nucleic acid. Other ways of identifying the sequence (e.g., the presence of a specific sequence) include, without limitation, amplification with gene/sequence specific primers (e.g., with real-time or quantitative PCR or a microarray (see, e.g., U.S. patent application Ser. No. 12/487,789).

In accordance with another aspect of the instant invention, in vitro assays are provided. The in vitro assays are preferable non-radioactive and allow for high-throughput sequencing. The in vitro methods employs the release of DNA from a protein (e.g., TOP2 cleavage complexes) by hydrolysis of phosphodiester bonds (e.g., with CIP) and high-throughput sequencing in order to map cleavage sites (e.g., TOP2 cleavage sites) in vitro with exact base precision. In a particular embodiment, the method comprises the following steps (exemplified with TOP2):

1. Obtaining a double stranded DNA substrate comprising a target sequence. In a particular embodiment, the substrate is obtained by PCR. In a particular embodiment, the double stranded DNA may be obtained by cloning a substrate (e.g., the entire 8.3 kb DNA fragment spanning the MLL bcr) into a plasmid, optionally treating the double stranded DNA plasmid substrate with T4 DNA ligase to assure that substrate is not nicked, releasing the insert (substrate) for analysis from the plasmid (e.g., by restriction enzyme cleavage), treating the released insert with phosphatase (e.g., CIP) to prevent re-ligation of the substrate after restriction enzyme cleavage, inactivating the phosphatase (e.g., by heat), and purifying the released double-stranded substrate (e.g., on a gel).

2. Subjecting the purified double stranded substrate to in vitro cleavage in the presence/absence of a TOP2 poison in a reaction mixture comprising TOP2 (e.g., TOP2A or TOP2B), ATP, and divalent cation (Mg²⁺) and, optionally, transferring reaction products to new buffer (e.g., cell lysis buffer).

3. Optionally fragmenting the DNA (e.g., into ˜500 bp segments (e.g., by sonication)) can be performed, although this step will typically be unnecessary for in vitro methods.

4. Isolating (e.g., by immunoprecipitating) TOP2 and TOP2 complexes such as DNA-bound TOP2. The immunoprecipitation may comprise adding α-TOP2 IgG to the sample to bind TOP2 (including DNA-bound TOP2) and immunoprecipitating α-TOP2 IgG containing complexes. For example, α-TOP2 IgG may be bound to Protein G magnetic beads and then the Protein G magnetic bead-bound fraction may be separated from non-bound fraction by using a magnet. The immunoprecipitation may be repeated on non-bound fraction more than once, optionally using fresh α-TOP2 IgG and Protein G each time. Bound fractions may be combined after the rounds of immunoprecipitation.

5. Treating the bound fraction from the immunoprecipitation with a phosphatase (e.g., calf intestinal phosphatase) to release TOP2-bound DNA from TOP2 cleavage complexes.

6. Determining the sequence of the released DNA. The sequence of the DNA may be determined by the methods described hereinabove.

In accordance with another aspect of the instant invention, kits for performing the methods of the instant invention are provided. In a particular embodiment, the kit comprises at least one, two, three, or all four of: a) a solid support and a buffer for isolating protein-nucleic acid complexes; b) a polymerase; c) a polynucleotide kinase; and d) a phosphatase. The kits may further comprise at least one of a) an agent and/or a buffer for lysing cells; b) at least one adaptor; c) at least one primer specific for said adaptor; d) antibody immunologically specific for the antibody of the protein of said protein-nucleic acid complex; e) instruction material; and f) any other component listed in the methods hereinabove.

As explained hereinabove, the methods and kits of the instant invention allow for the identification of nucleotide sequences in protein-nucleic acid complexes. The identification of the sequences allows for the prediction of where these complexes will form. The location (sequence) of these complexes will vary based on the presence or absence of particular agents. In the context of topoisomerase II, the topoisomerase II-DNA complexes will form at different sequences with different frequency based on the presence of different topoisomerase II poisons (e.g., different translocation events will also occur). Indeed, the identification of the sequences of the topoisomerase II-DNA complexes with various toposiomerase II poisons may be correlated with the likelihood, severity and/or frequency of secondary cancer (i.e., a screen of multiple patients). The identification of these sequences will allow for the rapid screening of those subjects/patients receiving topoisomerase II poisons as part of a chemotherapy regimen to provide a prognosis or risk assessment for developing a secondary cancer. In response to the identification of the sequences within the subject receiving the toposiomerase II poison, the treatment may be discontinued and/or aggressive, early screening, monitoring for disease occurrence, and, if indicated, treatment for a secondary cancer may be initiated.

### DEFINITIONS

As used herein, the term “microarray” refers to an ordered arrangement of hybridizable array elements. The array elements are arranged so that there are preferably at least one or more different array elements, more preferably at least 100 array elements, and most preferably at least 1,000 array elements on a solid support.

Preferably, the hybridization signal from each of the array elements is individually distinguishable, the solid support is a chip, and the array elements comprise oligonucleotide probes.

The term “MLL partner gene” refers to the gene or genomic DNA sequence fused with MLL after a translocation, such as those fusions present in certain leukemias.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

For instance, one common formula for calculating the stringency conditions required to achieve hybridization between nucleic acid molecules of a specified sequence homology is set forth below (Sambrook et al., 1989):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “primer” as used herein refers to a DNA oligonucleotide, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “isolated” may refer to a compound or complex that has been sufficiently separated from other compounds with which it was associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

As used herein, an “instructional material” includes a publication, a recording, a diagram, or any other medium of expression which can be used to communicate the usefulness of the composition of the invention for performing a method of the invention.

The phrase “solid support” refers to any solid surface including, without limitation, any chip (for example, silica-based, glass, or gold chip), glass slide, membrane, bead, solid particle (for example, agarose, sepharose, polystyrene or magnetic bead), column (or column material), test tube, or microtiter dish.

With respect to antibodies, the term “immunologically specific” refers to antibodies that bind to one or more epitopes of a protein or compound of interest, but which do not substantially recognize and bind other molecules in a sample containing a mixed population of antigenic biological molecules.

As used herein, the term “adaptor” refers to an oligonucleotide comprising a known sequence. The adaptor typically includes at least one site for primer binding. In some embodiments, adaptors of the invention have a length of about 10 to about 250 nucleotides, about 20 to about 200, or about 20 to about 100.

The following examples are provided to illustrate various embodiments of the present invention. They are not intended to limit the invention in any way.

### Example 1

### Methods

### Cell Culture for Maintenance of CCRF-CEM Cell Line

The cells utilized herein were CCRF-CEM cells. The CEM cell line is a T-cell lymphoblast-like cell line derived from a four-year old female Caucasian patient with T-cell ALL (Foley et al. (1965) Cancer, 18:522-529). This cell line cell has no consistent genetic aberrations (www.ATCC.com). Although it is desirous to use primary hematopoietic stem cells to model the cells targeted for translocations in vivo in patients, the CCRF-CEM cell line was used to establish the appropriate conditions. Cryo-preserved cells (40×10⁶) were thawed in a 37° C. water bath. Cells were then added to a 50 ml conical tube containing 25 ml of RPMI 1640 media (Invitrogen; Carlsbad, Calif.) and 10% heat inactivated fetal bovine serum (FBS) (10% FBS RPMI) (Thermo Scientific; Waltham, Mass.). After centrifugation at 1200 rpm for 5 minutes, media was aspirated and the cell pellet was resuspended in a 25 ml of fresh 10% FBS RPMI. Cells were transferred to a T75 flask and grown in a 37° C./5% CO₂ humidified incubator. Cells were maintained at 37° C./5% CO₂ in 10% FBS RPMI and passaged every three to four days.

### Isolation of Topoisomerase IIa (TOP2A) and TOP2A-Bound DNA

A T75 flask containing CEM cells was removed from the 37° C./5% CO₂ incubator. Cells were transferred from a T75 flask to a 50 ml conical tube and centrifuged at 1200 rpm for 5 minutes. Media was aspirated and the cell pellet was resuspended at 20×10⁶ cells in 10 ml of 10% FBS RPMI. Cells were centrifuged at 1200 rpm for 5 minutes. The supernatant was aspirated and the pellet was resuspended in 2 ml of Dulbecco's 1× Phosphate Buffered Saline (PBS) (Invitrogen) to wash out any remaining RPMI media. The resuspended cells were then centrifuged at 1200 rpm for 5 minutes and the supernatant was aspirated. The cell pellet contained in the 50 ml conical tube was now ready for lysis.

### Testing of Different Lysis Procedures for Recovery of TOP2A

In order to isolate topoisomerase IIα (TOP2A) including DNA-bound TOP2A, the nuclear contents first had to be obtained by lysing the cell and nuclear membranes of CEM cells using lysis buffers. Three different lysis procedures were tested in order to determine which buffer provided the best recovery of TOP2A. The first lysis procedure used Cell Membrane Lysis Buffer and Nuclear Membrane Lysis Buffer. These buffers are considered less harsh because of their nonionic properties. The second lysis procedure used CHAPS Lysis Buffer, which is mildly harsh because it has zwitterionic properties. The third lysis procedure used RIPA Lysis Buffer, which is a harsh buffer because of its ionic properties.

Protease inhibitor (PI), which prevents degradation of proteins, including TOP2A, by proteases, was prepared for addition to lysis buffers. One Complete™ Protease Inhibitor (PI), EDTA-free tablet (Roche; Basel, Switzerland) dissolved in 2 mL of dH₂0 in a 10 mL conical tube to make 25×PI stock. PI was stored at −20° C.

### Lysis Procedure #1 (Cell Membrane and Nuclear Membrane Lysis Buffers):

To make Cell Membrane Lysis Buffer, 0.2 mL of 5M stock of NaCl, 0.2 mL of Igepal CA-630 (Sigma; St. Louis, Mo.) and 1 mL of 1M stock of Tris-Base was reconstituted with NANOpure™ water for a final volume of 100 mL. The final concentrations were: 10 mM NaCl, 0.2% Igepal CA-630 and 10 mM Tris-Base. Cell Membrane Lysis Buffer (100 mL) was stored at 4° C. When needed, 10 mL of Cell Membrane Lysis Buffer were transferred to a 15 mL conical tube. The 25× stock of PI was thawed and 400 μL was added to the 10 mL of Cell Membrane Lysis Buffer (final concentration of PI=1×).

To make Nuclear Membrane Lysis Buffer, 2.5 mL of 1M stock of Tris-Base, 1.0 mL of 0.5M stock of EDTA (Invitrogen) and 5.0 mL of 10% stock of SDS (Invitrogen) was reconstituted with NANOpure™ water for a final volume of 50 mL. The final concentrations were 50 mM Tris-Base, 10 mM EDTA and 1% SDS. Nuclear Membrane Lysis Buffer (50 mL) was stored at room temperature. When needed, 10 mL of Nuclear Membrane Lysis Buffer were transferred to a 15 mL conical tube. The 25× stock of PI was thawed and 400 μL was added to the 10 mL of Nuclear Membrane Lysis Buffer (final concentration of PI=1×).

Cell membranes were lysed by adding 500 μL of Cell Membrane Lysis Buffer containing 1×PI to the conical tube containing the cell pellet. Following lysis, the cell lysate was transferred to a microcentrifuge tube and incubated on ice for 15 minutes, with vortexing every 5 minutes. The cell lysate was then centrifuged at 4° C. for 15 minutes at 13,200 rpm to separate the nuclei. The supernatant (non-nuclear components of cell lysate) was placed into a new microcentrifuge tube and stored at −20° C. The nuclei (contained in the pellet) were lysed with 500 μL of Nuclear Membrane Lysis Buffer containing 1×PI. To ensure maximal lysis, the nuclear lysate was pulled through 18-gauge, 22-gauge, 25-gauge and 27-gauge needles four times, respectively. The nuclear lysate contained total nuclear genomic DNA and proteins, including DNA-bound TOP2A. The microcentrifuge tube containing the nuclear lysate was placed on ice and immediately carried to the sonicator for fragmentation.

### Lysis Procedure #2 (CHAPS Lysis Buffer):

To prepare CHAPS Lysis Buffer, 2.74 mL of 5M stock of NaCl, 10 mL of 100% Glycerol, 2 g of CHAPS powder (Sigma), 0.4 mL of 0.5M EDTA and 2 mL of 1M stock of Tris-Cl (pH 7.5) was reconstituted in NANOpure™ water for a final volume of 100 mL (final concentrations: 137 mM NaCl, 10% glycerol, 2% CHAPS, 2 mM EDTA, 20 mM Tris-Cl). CHAPS Lysis Buffer (100 mL) was stored at 4° C. When needed, 10 mL of CHAPS Lysis Buffer were transferred to 15 mL conical tube. The 25×PI was thawed and 400 μL was added to 10 mL of CHAPS Lysis Buffer (final PI=1×).

First, 500 μL of CHAPS Lysis Buffer containing 1×PI was added to the cell pellet to lyse the cell and nuclear membranes of CEM cells. The whole cell lysate was then transferred to a microcentrifuge tube followed by a 15-minute incubation on ice, with vortexing every 5 minutes. The whole cell lysate was then pulled through 25-gauge and 27-gauge needles, four times each, to ensure maximal lysis (higher gauge needles used since CHAPS is a harsher buffer, because of its zwitterionic properties, compared to Cell or Nuclear Membrane Lysis Buffers). Lysate was centrifuged at 4° C. for 20 minutes at 13,200 rpm. The supernatant (whole cell lysate) was placed in a new microcentrifuge tube and the pellet discarded. The whole cell lysate contained total cellular genomic DNA and proteins, including DNA-bound TOP2A. The microcentrifuge tube containing whole cell lysate was placed on ice and immediately carried to the sonicator for fragmentation.

### Lysis Procedure #3 (RIPA Lysis Buffer):

To make RIPA Lysis Buffer, 3 mL of 5M stock of NaCl, 1 mL of Igepal CA-630, 1 g Na deoxycholate, 5 mL of 10% stock of SDS and 5 mL of 1M stock of Tris-HCl (pH 7.2) was reconstituted in NANOpure™ water for a final volume of 100 mL or proportional amounts of reagents to make 500 mL. The final concentrations were 150 mM NaCl, 1% Igepal CA-630, 1% Na deoxycholate, 0.5% SDS and 50 mM Tris-HCl. When needed, 10 mL of RIPA Lysis Buffer were transferred to a 15 mL conical tube. The 25× stock of PI was thawed and 400 μL was added to the 10 mL of RIPA Lysis Buffer (final PI=1×).

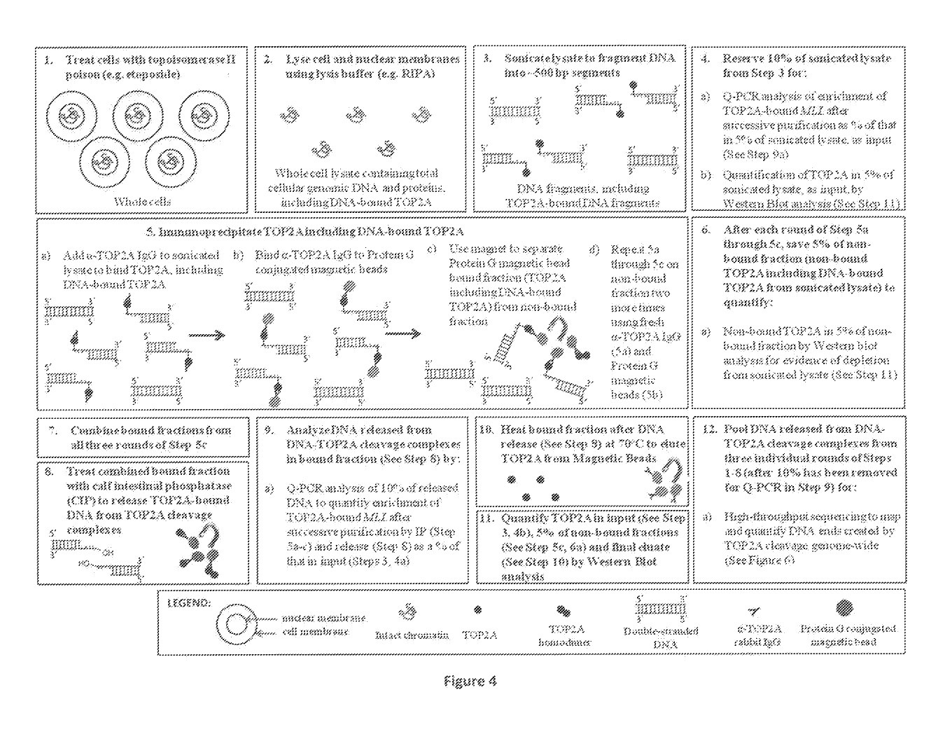

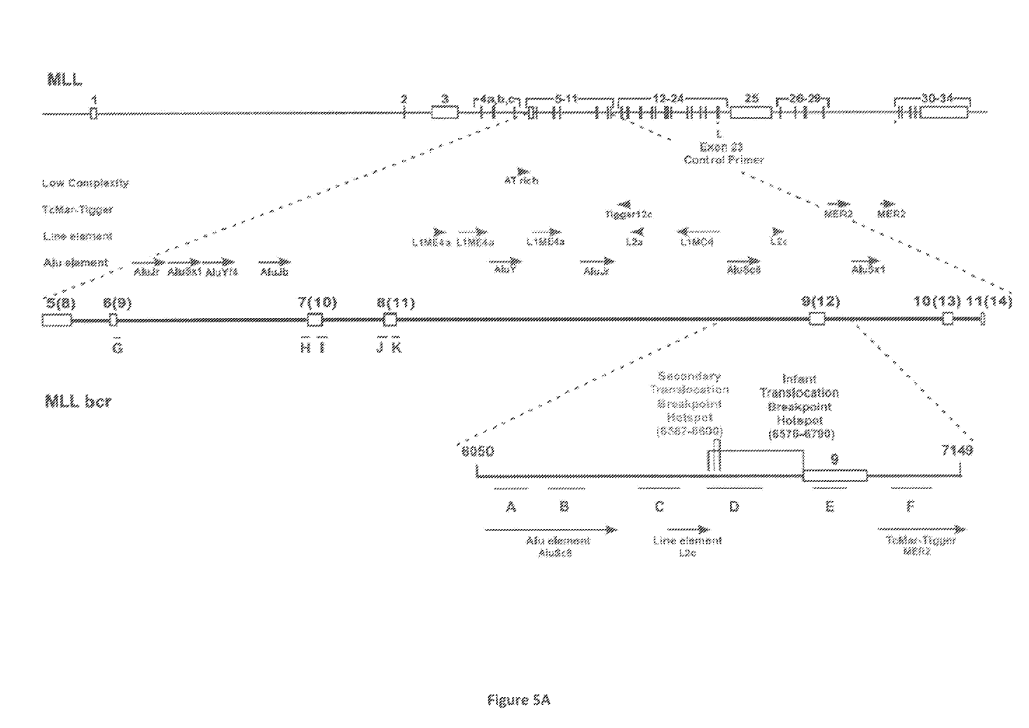

First, 500 μL of RIPA Buffer containing 1× PI was added to the cell pellet to lyse the cell and nuclear membrane. The whole cell lysate was then transferred to a microcentrifuge tube followed by a 15-minute incubation on ice, with vortexing every 5 minutes. The lysate was then pulled through 25-gauge and 27-gauge needles, four times each, to ensure maximal lysis (higher gauge needles were used since RIPA is a harsh buffer, because of its ionic properties, compared to Cell Membrane or Nuclear Membrane Lysis Buffer). The lysate was centrifuged at 4° C. for 20 minutes at 13,200 rpm. The supernatant (whole cell lysate) was placed in a new tube and the pellet discarded. The whole cell lysate contained total cellular genomic DNA and proteins, including DNA-bound TOP2A (See FIG. 4, Step 2). The microcentrifuge tube containing the whole cell lysate was placed on ice and immediately carried to the sonicator for fragmentation.

### DNA Fragmentation

In order to produce TOP2A-bound DNA fragments that were a desirable size for subsequent high-throughput sequencing, DNA was fragmented into segments of approximately 500 base pairs (bp). This was achieved through sonication with the Bioruptor® (Diagenode; Denville, N.J.), which used ultrasonic wave frequencies of 20-30 kHz (preset) to shear the DNA contained in the lysate (See FIG. 4, Step 3). Conditions were set for the sonicator to be on for 30 seconds and off for 2 minutes, in cycles, for a total of 15 minutes at 4° C. The 15 minute sonication was performed three times (with no time in between), for a total of 45 minutes.

Following sonication, 10% of the sonicated lysate was set aside (See FIG. 4, Step 4). Five percent of this will be used as “input” in Q-PCR analysis and was stored at −20° C. The other 5% will be used as “input” in Western blot analysis for quantification of TOP2A and was stored at 4° C. The remaining 90% of the sonicated lysate was used for immunoprecipitation. Depending on the experiment, the sonicated lysate may have been stored overnight at −20° C. before immunoprecipitation.

### Immunoprecipitation (IP) of TOP2A Including DNA-Bound TOP2A

In order to immunoprecipitate TOP2A and DNA-bound TOP2A, 10 μL (1 μg/1 μL stock×10 μL=10 μg) of α-TOP2A polyclonal rabbit IgG (Kamiya; Seattle, Wash.) or 10 μL of α-TOP2A (3F6) monoclonal mouse IgG1 (Santa Cruz; Santa Cruz, Calif.) was added to the sonicated lysate (See FIG. 4, Step 5a). Alternatively, either 10 μL (2.5 μg/1 μL stock×10 μL=2.5 μg) of α-eIF4E monoclonal mouse IgG1 (BD Transduction Laboratories; Franklin Lakes, N.J.) or 50 μL (0.2 μg/1 μL stock×50 μL=10 μg) of α-BECN1 polyclonal IgG (H-300) rabbit IgG (Santa Cruz) as the negative control antibody, was added to the lysate. The antibody and lysate mixture rotated at 4° C. for one hour.

During this time, 50 μL of PureProteome™ Protein G magnetic beads (Millipore; Billerica, Mass.) were prepared for each lysate in a new microcentrifuge tube for use in binding primary antibody. Since beads would accumulate at the bottom of the container they were delivered in, the container was first shaken before transferring 50 μL to the microcentrifuge tube. Following this, the storage buffer (aqueous benzyl alcohol) was removed by using the magnetic Magna GrIP™ Rack (Millipore), so that beads would immediately adhere to the side of the tube and separate from the storage buffer. A wash solution (50 mL) was made consisting of 49.95 mL of PBS and 50 μL of Tween® 20 surfactant (Fischer Scientific; Waltham, Mass.) (final concentration: 0.1% Tween® 20). The beads were washed with 500 μL of this wash solution and vortexed for 10 seconds. The magnet was used to separate the beads and the supernatant was discarded.

After the primary antibody and sonicated lysate mixture had incubated for one hour, the mixture was added to the microcentrifuge tube containing the Protein G magnetic beads (See FIG. 4, Step 5b). The tube was placed into a styrofoam holder shaken by hand for 10 minutes at room temperature to ensure mixing. 10 minute v. 30 minute v. overnight incubation with beads was compared. The magnet was used to separate Protein G magnetic bead bound fraction (containing TOP2A, including DNA-bound TOP2A) from the supernatant, or non-bound fraction (See FIG. 4, Step 5c). The non-bound fraction was placed into a new microcentrifuge tube and stored at 4° C. for use in Western blot for analysis of TOP2A depletion from the sonicated lysate (See FIG. 4, Step 6). The bound fraction was washed three times with 500 μL of wash solution to minimize nonspecific binding to the beads. For each wash, the magnet was used to separate the bound fraction from the supernatant (anything non-specifically bound to the beads) which was discarded.

### Additional Depletion Using Multiple Rounds of Immunoprecipitation:

Additional experiments tested whether measures could be taken to further deplete remaining TOP2A from the non-bound fraction in an effort to establish a quantitative assay. In experiments below where more than one round of IP was done, 5% of non-bound fraction was transferred to a new tube and stored at −20° C. to be used in Western blot analysis for evidence of depletion of TOP2A. For the additional IP rounds, fresh 10 μg of α-TOP2A rabbit IgG (Kamiya) followed by fresh 50 μL of Protein G magnetic beads were added to the remaining 95% of non-bound fraction.

In one experiment, it was tested whether two rounds of immunoprecipitation would increase TOP2A depletion from the non-bound fraction. After the non-bound fraction was obtained from the first round of IP, 5% was placed in a new tube and stored at −20° C. for use in Western blot analysis for evidence of TOP2A depletion from the sonicated lysate. The remaining 95% of the non-bound fraction was also stored overnight at −20° C., since this was a convenient stopping point. The following day, fresh α-TOP2A rabbit IgG followed by fresh Protein G magnetic beads were added to the remaining 95% of the non-bound fraction. In this experiment, the two rounds of beads were not combined. Therefore, two separate non-bound fractions and two separate bound fractions were obtained.

Another experiment tested whether using just one round of IP after freezing of the sonicated lysate at −20° C. would increase depletion of TOP2A from the non-bound fraction. After overnight freezing, the sonicated input was thawed followed by IP using one round of α-TOP2A rabbit IgG and Protein G magnetic beads. A single non-bound fraction and bound fraction were obtained.

In another experiment, it was tested whether three rounds of immunoprecipitation (see FIG. 4, Step 5d) after overnight storage of the sonicated input at −20° C. would increase depletion of TOP2A from the non-bound fraction. After overnight storage, the sonicated input was thawed followed by IP using α-TOP2A rabbit IgG and Protein G magnetic beads. After the non-bound fraction (#1) was obtained and placed into a new microcentrifuge tube, 5% was transferred to a new tube and stored at −20° C. to be used in Western blot analysis for evidence of depletion of TOP2A from the sonicated lysate. The bead-bound fraction was placed on ice. Fresh α-TOP2A rabbit IgG followed by fresh Protein G magnetic beads were added to the remaining 95% of the non-bound fraction (#1) for a second round of IP.

Once the non-bound fraction (#2) was obtained, 5% was transferred to a new microcentrifuge tube and stored at −20° C. The bound fraction (#2) was placed on ice.

Fresh α-TOP2A rabbit IgG followed by fresh Protein G magnetic beads were added to the remaining 95% of the non-bound fraction (#2) for a third round of IP. The final non-bound fraction (#3) was obtained and stored at −20° C. The final bound fraction (#3) was placed on ice. All three bound fractions were then removed from ice and resuspended in 250 μL of PBS containing 0.1% Tween® 20 surfactant. The three sets of resuspended bound fractions were then combined into one microcentrifuge tube as the “combined bound fraction” (See FIG. 4, Step 7). Therefore, in this experiment, three non-bound fractions and one combined bound fraction were obtained.

Release of TOP2A-bound DNA from TOP2A Cleavage Complexes

In order to analyze the TOP2A-bound DNA in the bound fraction, it first had to be released from the covalent bond with TOP2A using calf intestinal phosphatase (CIP) (10,000 units/mL stock) (New England Biolabs; Ipswich, Mass.). CIP releases the phosphate groups from phosphorylated tyrosine, serine and threonine residues in proteins (Sambrook et al. (1989) Molecular Cloning: A Laboratory Manual. New York: Cold Spring Harbor Laboratory Press). Since DNA is covalently bound to TOP2A by means of a phosphotyrosyl bond, this activity would release TOP2A-bound DNA from TOP2A.

Combined bound fraction was washed two times with 1×NEBuffer 3 (10× stock diluted to 1× with dH₂0) (Final concentrations: 100 mM NaCl, 50 mM Tris-HCl, 10 mM MgCl₂, 1 mM Dithiothreitol pH 7.9) (NE Biolabs) at a volume equal to the total μL of Protein G magnetic beads used (i.e. if one round of IP was done with a total of 50 μL of beads, 50 μL of 1×NEBuffer 3 was used; if two rounds of IP were done with a total of 100 μL of beads, 100 μL of NEBuffer 3 was used, etc.). The bound fraction was separated after each wash with the magnet and the supernatant discarded.

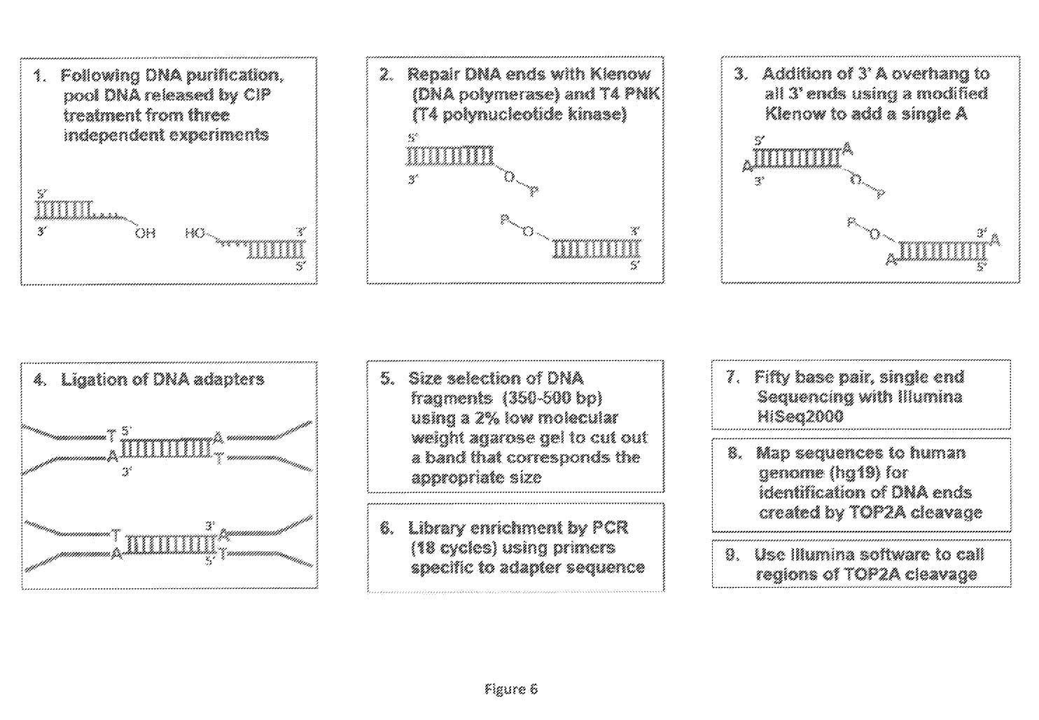

The combined bound fraction was added to a solution containing 88% dH₂0, 10% of 10×NEBuffer 3 (final concentration: 1×) and 2% CIP (final concentration: 200 units/mL) following incubation for one hour in a 37° C. water bath (See FIG. 4, Step 8). The total volume used for the CIP incubation was equal to the total μL of Protein G magnetic beads used. For example, when 50 μL of beads were used during IP, the total volume for CIP incubation was 50 μL, of which 1 μL was CIP (10,000 units/mL×1 μL=10 units). Since the beads tended to accumulate at the bottom, the microcentrifuge tubes were shaken by hand every fifteen minutes to ensure proper mixing. After one hour, the mixture was removed from the water bath and placed on the magnetic rack. The supernatant (DNA released from DNA-TOP2A cleavage complexes in combined bound fraction) was transferred to a new microcentrifuge tube and stored at 4° C. Ten percent of the released DNA will be used in Q-PCR analysis to quantify enrichment of TOP2A-bound MLL as a percentage of input (See FIG. 4, Step 9a). The remaining 90% of the released DNA will be used in high-throughput sequencing to map and quantify DNA ends created by TOP2A cleavage genome-wide (See FIG. 4, Step 12 and FIG. 6).

Elution of TOP2A from Protein G Magnetic Beads

TOP2A was eluted from Protein G magnetic beads by heating the sample at 70° C. for 15 minutes in a solution containing 1×LDS Sample Buffer (Invitrogen) and 5% β-mercaptoethanol (β-ME) (BioRad; Hercules, Calif.), both of which prevent the formation of disulfide bonds. The volume that the TOP2A was eluted in varied. If one or two rounds of IP were done, then a total volume of 25 μL was used for elution. If three rounds of IP were done, then a total volume of 40 μL was used for elution. After 15 minutes at 70° C., the magnet was used to separate the beads and the supernatant (eluted TOP2A) was transferred to a new tube, as the eluate, and stored at −20° C. for use in the quantification of TOP2A contained in the bound fraction by Western blot analysis (See FIG. 4, Steps 10 and 11).

### Demonstration of TOP2A by Western Blot Analysis

Western blot analysis was utilized to quantify TOP2A in the sonicated lysates, non-bound fractions and eluates (See FIG. 4, Step 11). In order to prepare samples for the Western blot, the input (5% of sonicated lysate) and 5% of the non-bound fractions, both in 1×LDS and 2.5% β-ME (final volume of 20 μL), were incubated at 70° C. for 15 minutes. The final non-bound fraction volume is the volume of the sonicated lysate plus the volume of antibody added (see Step 5a of FIG. 4). Previously frozen eluates were removed from the freezer, thawed and incubated at 70° C. for 15 minutes.

The apparatus used for the Western Blot was the X Cell II™ Blot Module (Invitrogen). Either a 12-lane 3-8% Tris-Acetate Gel (Invitrogen) or a 10-lane 7% NuPAGE® Tris-Acetate Gel (Invitrogen) was used. The full 20 μL of each prepared sample were loaded into its assigned lane. The HiMark™ Pre-Stained Protein Standard (Range: 31 kDa to 460 kDa) (Invitrogen) or the Novex® Sharp Pre-Stained Protein Standard (Range: 3.5 kDa to 260 kDa) (Invitrogen) was used as a measure of protein size. These standards were selected because their range encompassed the 170 kDa size of TOP2A.

Outer chamber running buffer was made by combining 50 mL of Novex®Tris-Acetate SDS Running Buffer (Invitrogen) (final concentration: 5%) and 950 mL of NANOpure™ water. To make inner chamber running buffer, 200 mL of outer chamber buffer was transferred to a new flask and 0.5 mL of NuPAGE® antioxidant (Invitrogen; Catalog #: NP0005) (final concentration: 0.25%) was added. Following this, ˜190 mL of inner chamber running buffer was added to the inner chamber and ˜750 mL of outer chamber running buffer was added to the outer chamber. Samples were electrophoresed at 150 volts for 70 minutes, allowing for separation of marker along the length of the gel.

Proteins were transferred from the gel onto a methanol-activated Invitrolon™ PVDF Filter Paper Sandwich with a 0.45 micron pore size (Invitrogen) for 2 hours at 25 volts in the X Cell II™ Blot Module apparatus (Invitrogen). The inner chamber transfer buffer was made by adding 50 mL of 20×NuPAGE® Transfer Buffer (Invitrogen) (final concentration: 5% or 1×), 100 mL of methanol (final concentration: 10%), 1 mL of NuPAGE® antioxidant (final concentration: 0.1%) and 849 mL of NANOpure™ water. Once buffers were made, ˜25 mL of the transfer buffer was added to the inner chamber and ˜750 mL of water was added to the outer chamber.

Following the transfer, blocking solution was prepared. TBS-Tween® (TBST) was made by adding 10 mL of 1M Tris-HCl pH 7.5, 30 mL of 5M NaCl and 500 μL of Tween® 20 surfactant to 960 mL of water to give a final volume of 1 L and final concentrations of 10 mM Tris-HCl pH 7.5, 150 mM NaCl, 0.05% Tween® 20 surfactant. Blocking solution was made by adding 5% (w/v) non-fat dry milk (NFDM) and 1 mL of 2% (w/v) NaN₃ in 100 mL TBS-Tween®. Ten mL of the blocking solution was incubated on the blot for 1 hour at room temperature on a rocker. The blocking solution was drained from the plate and the blot was then rinsed with 10 mL of TBS-Tween®. This wash was drained from the plate followed by overnight incubation of the blot with primary α-TOP2A (3F6) monoclonal mouse IgG1 (Santa Cruz) or primary rabbit α-TOP2A polyclonal rabbit IgG (Kamiya) in a 1:500 (α-TOP2A IgG: TBST) solution, containing 1% NFDM, in a 4° C. cold room on a rocker.

The following day, the blot was washed three times in 10 mL of TBST. Each wash lasted 5 minutes on a rocker and the TBST was drained from the plate before the next wash began. Following the last wash, the TBST was drained from the plate and the blot was then exposed to secondary horseradish peroxidase-linked anti-mouse IgG1 (from sheep) (GE Healthcare; Piscataway, N.J.) or secondary horseradish anti-rabbit IgG1 antibody (from donkey) (GE Healthcare) in a 1:2000 (secondary IgG: TBST) solution, containing 0.05% NFDM, for 1 hour at room temperature on a rocker. This allowed for the secondary antibody to bind the primary antibody. After three additional washes with TBST, Amersham™ ECL Plus Western Blotting Detection System (GE Healthcare) was utilized to detect the proteins by chemiluminescence. The blots were then placed into an autoradiography cassette (Fischer). The blots were immediately exposed to film in a dark room. The film was then developed using the Kodak X-OMAT 2000 Processor.

Analysis of Input DNA and DNA Released from TOP2A-DNA Complexes

The volume containing DNA released from DNA-TOP2A cleavage complexes by CIP treatment (See FIG. 4, Step 8) was brought to 200 μL by adding dH₂0. In addition, the input (5% of sonicated lysate) was brought to 200 μL with dH₂0. All samples then underwent an equal volume extraction with Phenol:Chloroform (1:1) (Applied Biosystems) followed by centrifugation at 14,000 rpm for 2 minutes. The top aqueous layer (containing DNA) was transferred to a new microcentrifuge tube and the bottom layer was discarded. This was followed by an equal volume extraction with Chloroform (Sigma) and centrifugation at 14,000 rpm for 2 minutes. The top layer (containing DNA) was transferred to a new microcentrifuge tube. Following the chloroform extraction, 10 μL of 3M NaOAc (pH 7.0) (final concentration: 143 mM) was added prior to 400 μL of ice cold 100% ethanol added to the top layer and vortexed. In order to make the final NaOAc concentration 300 mM, 22 μL (rather than 10 μL) of 3M NaOAc will be used. Samples were incubated on dry ice for 10 minutes followed by centrifugation at 13,200 rpm for 30 minutes at 4° C. The supernatant was removed and the DNA pellet was washed with 750 μL of ice cold 100% ethanol. Following centrifugation at 13,200 rpm for 5 minutes at 4° C. and disposal of the supernatant, the pellet was washed with 750 μL of ice cold 70% ethanol. After this, samples were again centrifuged at 13,200 rpm for 5 minutes at 4° C. and the supernatant was removed. The pellet was then desiccated for 5 minutes in the Savant Speed Vac Concentrator SVC 100H to fully dry the purified DNA. The DNA was resuspended in 20 μL of dH₂0 followed by incubation at room temperature for 20 minutes to ensure resuspension. The purified DNA was stored at 4° C.

In order to determine whether sonication had properly fragmented DNA into segments of 500 bp or less, input DNA was electrophoresed. TAE (Tris-Acetate-EDTA) (40×) was stored at room temperature and consisted of 194.4 g Tris, 108.9 g NaOAc, 15.2 g EDTA and pH stabilized to 7.2 using approximately 80 mL acetic acid to achieve a total volume of 1 L and final concentrations of 1.6 M Tris, 1.25 M NaOAc and 4 mM EDTA. This was diluted with water (250 mL of 40×TAE in 9.75 L of water) to prepare a 1× working stock with final concentrations of 40 mM Tris, 31.25 mM NaOAc and 0.1 mM EDTA. To prepare the 2% agarose gel, 50 mL of 1× TAE was added to a 125 mL flask. One gram of agarose (Invitrogen) was weighed and added to the flask with TAE. The mixture was heated in a microwave for 2 minutes to dissolve the agarose. After this time, cold water was run over the bottom of the flask for 30 seconds to cool it. Following this, 3.3 μL of ethidium bromide (Sigma) was added to the flask. Ethidium bromide is a fluorescent dye used to stain nucleic acids. The mixture was swirled to distribute evenly and then 20 mL was poured into a gel chamber. A comb was placed at one end of the box into the mixture to make six lanes. After approximately 30 minutes, the mixture had formed into a solid gel. The gel was transferred to an electrode box and 1×TAE was poured to cover the gel completely.

Purified DNA from the input (5% of sonicated lysate) was removed from storage at 4° C. On a piece of parafilm, 5 μL of input DNA was added to 5 μL of dH₂0 and 2 μL of 6×DNA Loading Dye (Fermentas). The full 12 μL of the mixture was loaded into a lane in the gel. In addition, 5 μL of Gene Ruler 1 kb DNA Ladder Plus (Range: 20,000 bp to 75 bp) (Fermentas; Glen Burnie, Md.) was loaded into an adjacent lane. This was used as a marker of DNA length. The DNA was electrophoresed for 40 minutes at 90 volts. After this time, the gel was removed from the electrode box and placed inside the Gel Doc™ XR+(BioRad) to take a photograph of the gel to display the fluorescence of the ethidium bromide-stained DNA.

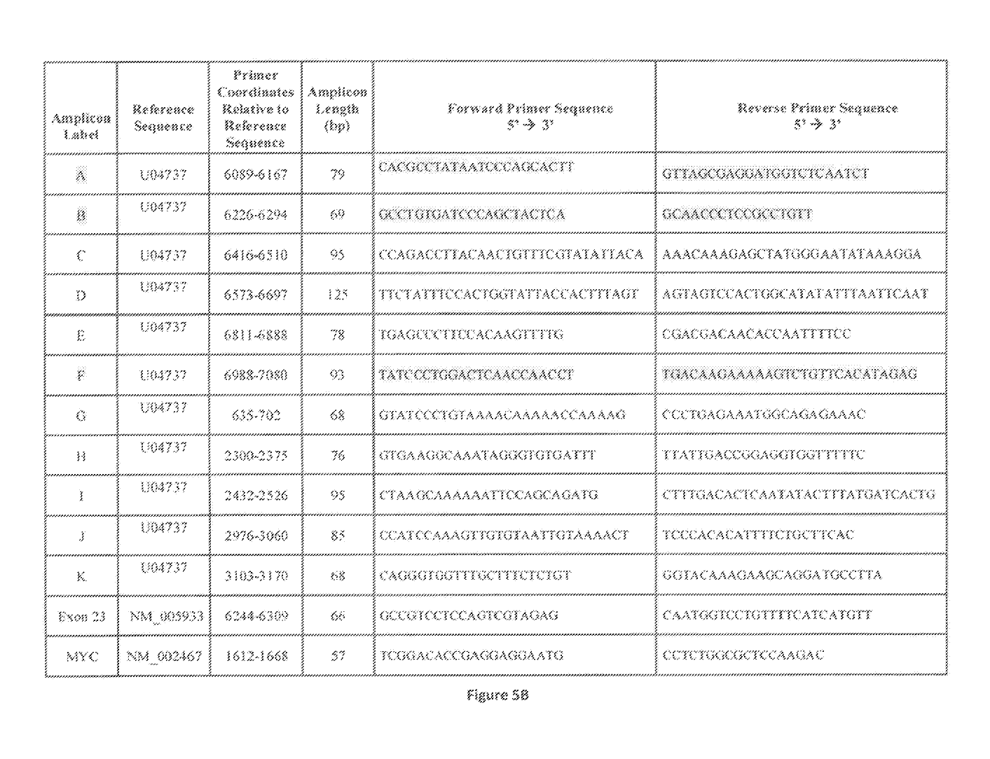

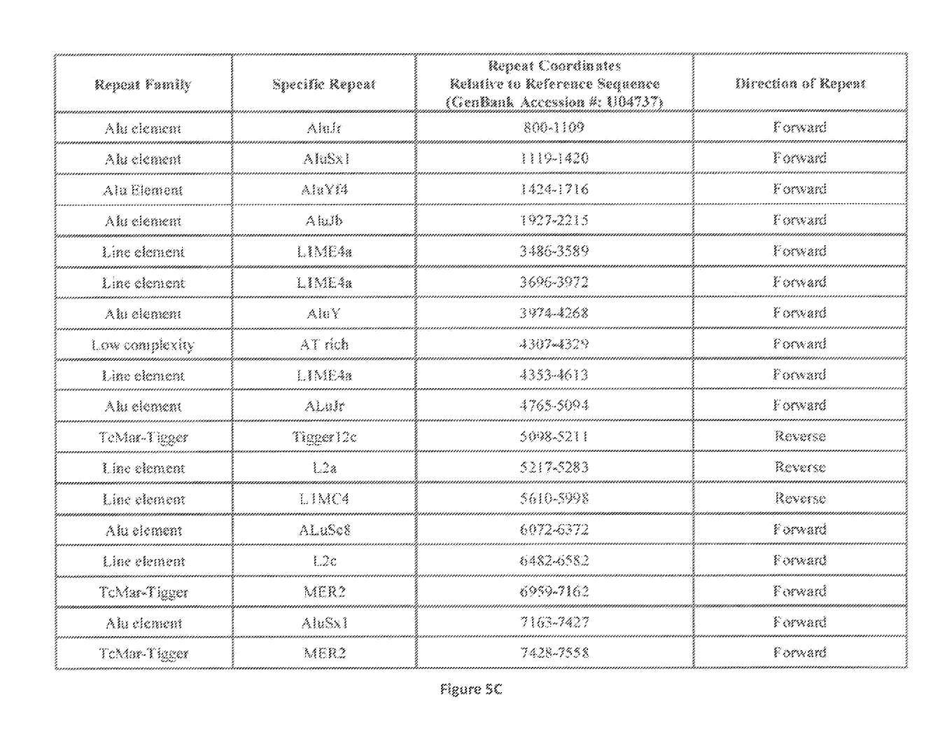

Quantitative Real-Time Polymerase Chain Reaction (Q-PCR) analysis was utilized to quantify enrichment of TOP2A-bound MLL as a percentage of the input (See FIG. 4, Steps 4a and 9a). Primer pairs were selected so that amplicons spanned the MLL bcr, preferably crossing intron-exon junctions to increase specificity, since repeats are commonly found within introns (See FIG. 5a). Notably, there are two numbering systems for introns/exons in MLL. The first number is Rasio et al. designation (Rasio et al. (1996) Cancer Res., 56:1766-1769.), whereas the parenthetical number is Nilson et al. designation (Nilson, et al. (1996) Br. J. Haematol., 93:966-972). A concentration of amplicons (A-F) was designed 3′ within intron 8(11) because of previous evidence that MLL translocation breakpoints in patients with leukemia occur with biased hotspots in this region even though they are heterogeneously distributed in the MLL bcr. More specifically, in secondary leukemia, a translocation breakpoint hotspot region has been shown in previous studies to span bases 6587-6600 (GenBank Accession #: U04737) in the MLL bcr (Megonigal et al. (2000) Proc. Natl. Acad. Sci., 97:2814-2819; Langer et al. (2003) Genes Chromosomes Cancer, 36:393-401; Megonigal et al. (1997) Proc. Natl. Acad. Sci., 94:11583-11588; Domer et al. (1995) Leukemia, 9:1305-1312). In addition, a breakpoint hotspot region in infants with leukemia has been shown to span bases 6576-6790 (GenBank Accession #: U04737) (Gillert et al. (1999) Oncogene, 18:4663-4671; Langer et al. (2003) Genes Chromosomes Cancer, 36:393-401; Leis et al. (1998) Leukemia, 12:758-763; Raffini et al. (2002) Proc. Natl. Acad. Sci., 99:4568-4573). Amplicons A-F were designed to flank regions around these breakpoint hotspots.

Primers were selected using the Primer Express® 3.0 Primer Probe Test Tool program using TaqMan® MGB settings and default parameters. This program selects compatible primer pairs based on their % GC content and melting temperatures. Primers were ordered from Integrated DNA Technologies (Coralville, Iowa) (See FIG. 5b for primer sequences). In addition to primers within the MLL bcr, two additional primer pairs were made as controls. The first control primer was MLL exon 23 (Rasio et al designation), which is not within the MLL bcr (See FIGS. 5a and 5b) and is not involved in translocations. The second control primer was within exon 3 of MYC, a gene that is not involved in MLL translocations (See FIG. 5b).

The location of all repetitive sequence elements within the MLL bcr was determined by using the RepeatMasker program (www.repeatmasker.org) (See FIGS. 5a and 5c). This was performed to compare primer pair sequences and amplicons to repeat elements in the MLL bcr (GenBank Accession #: U04737). Information about the specificity of these primer pairs to amplify selected amplicons in the bcr was necessary to ensure that the Q-PCR would only amplify the intended DNA within the MLL bcr, rather than a region of DNA repeated throughout the genome. Since primer pairs A, B and F were completely contained within repeats, additional testing was warranted to determine specificity of these primer pairs to the MLL bcr. BLAT (genome.ucsc.edu/cgi-bin/hgBlat) and BLAST (blast.ncbi.nlm.nih.gov/Blast.cgi) programs were used to test if primer pairs A, B and F were unique or if sequences were repeated in the human genome. These tests showed that B and F had either unique sequences or sequence divergence. Therefore, it was concluded that these primer pairs would produce a unique, specific product in the MLL bcr.

When the primers were first delivered, they were diluted with dH₂0. The number of μL of dH₂O added was equal to the number of nanomoles of each primer, so that the final concentration of each suspended primer was 1 nm/μL. Following this, a solution was made containing both primers in a primer pair so that the final concentration of each primer was 9 μM. To do this, 109 μL of dH₂0 was added to a new microcentrifuge tube. One μL of each primer (1 nm/μL×1 μL=1 nm) in the primer pair was then added to the dH₂0 so that the final volume was 111 μL. This achieved a final concentration of 9 μM for each primer (1 nm/111 μL=9 μM). Suspended primer pairs were stored at −20° C.

Purified DNA released from TOP2A-DNA cleavage complexes in bound fraction, purified DNA from α-BECN1 IgG (negative control) bound fraction and purified input DNA were loaded onto 384-well plates. Each well (10 μL total reaction volume) contained 1 μL of the primer pair, 5 μL of SYBR® Green PCR Master Mix (Applied Biosystems; Carlsbad, Calif.) and between 0.1 and 0.5 μL of DNA (depending on the experiment), with the remainder dH₂0. The plate was covered with an Optical Adhesive Cover (Applied Biosystems) and centrifuged at 1200 rpm for 1 minute. The Q-PCR computer program: SDS (Sequence Detection Systems) 2.3 was used and standard curve (absolute quantification), 384-well settings were used. Default Q-PCR cycling conditions were: 2 minutes at 50° C., 10 minutes at 95° C. followed by 40 cycles of: 15 seconds at 95° C. and 2 minutes at 60° C. A dissociation cycle (15 seconds at 95° C., 15 seconds at 60° C., 1 minute at 95° C.) was added to the end of the default settings, which is necessary when using SybrGreen® primers. The 384-well plate was run on an Applied Biosystems 7900 Real Time-PCR machine.

High-throughput sequencing may be used, particularly after topoisomerase II poison treatment. Both treated and non-treated samples may be sequenced. Purified DNA released by CIP treatment from DNA-TOP2A cleavage complexes in the bound fraction from three independent experiments will first be pooled (See FIG. 6, Step 1). In addition, DNA obtained from the bound fractions that are incubated with the negative control antibody, α-BECN1 IgG, will also undergo sequencing after pooling samples from three independent experiments. Pooling will control for biological diversity and improve the statistical power of the analysis. After pooling, DNA ends will be repaired. The DNA polymerase, Klenow, will add complementary bases to the strand opposite each of the four-base 5′ overhangs. T4 polynucleotide kinase, T4 PNK, will add a phosphate group to the 5′ OH residue at the 3′ side of cleavage on each strand that was introduced after CIP treatment (See FIG. 6, Step 2). Following this, there will be the addition of an adenine (A) overhang to all 3′ ends using a modified Klenow that will only add a single A to each 3′ end (See FIG. 6, Step 3). Ligation of DNA adapters will then take place by targeting the 3′ A overhangs. The adapter contains a complementary thymine (T) on the 5′ end (See FIG. 6, Step 4). Agarose gel-mediated size selection will isolate fragments that are 350-500 bp long. Specifically, a 2% low molecular weight agarose gel will be used in excising a band that corresponds to the appropriate size fraction after separation by electrophoresis (See FIG. 6, Step 5). Library amplification will be done by PCR (15 cycles) using primers that have been designed specific to the adapter sequences on both ends of the DNA molecules (See FIG. 6, Step 6). Single end, fifty base pair high-throughput sequencing will then be done using the Illumina (San Diego, Calif.) HiSeq2000 (See FIG. 6, Step 7). The sequences obtained will then be mapped to the human genome (hg19) in order to identify DNA ends created by TOP2A cleavage (See FIG. 6, Step 8). Illumina software will then be used to call regions of TOP2A cleavage (See FIG. 6, Step 9).

### Results

The following results are with regard to experiments that determine experimental conditions to 1) isolate and purify native TOP2A cleavage complexes (cleavage complexes that form in the absence of poison), 2) release and purify TOP2A-bound DNA from native TOP2A cleavage complexes by CIP treatment, 3) quantify MLL bcr sequences in the released, purified DNA by Q-PCR to validate enrichment before embarking on the sequencing and 4) quantify TOP2A obtained in the bound fraction compared to the non-bound fraction by Western blot analysis to determine whether immunodepletion of the sonicated lysate was achieved.

Isolation of TOP2A and TOP2A-bound DNA from Native TOP2A Cleavage Complexes
Testing Effects of Different Lysis Buffers on Achievement of Immunodepletion of TOP2A from the Sonicated Lysate

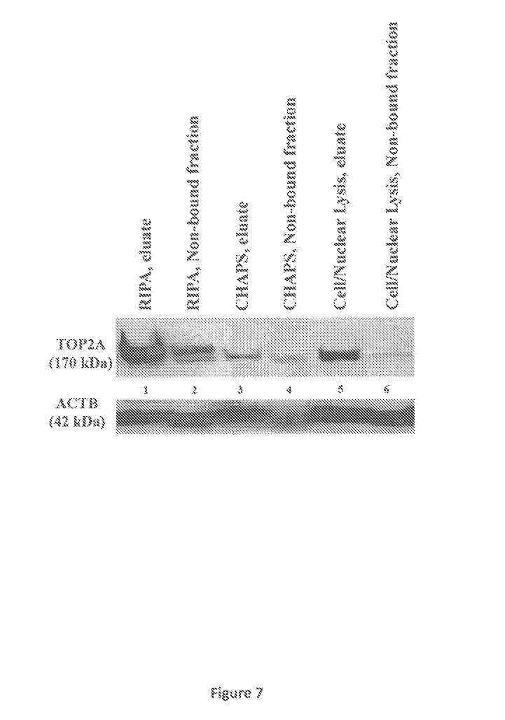

The first step towards optimizing TOP2A and TOP2A-bound DNA isolation was to choose a lysis buffer that would yield substantial TOP2A recovery after immunodepletion of the sonicated lysate. A comparison was made of RIPA Lysis Buffer, CHAPS Lysis Buffer and the combination of Cell Membrane Lysis Buffer followed by Nuclear Membrane Lysis Buffer. When the amount of TOP2A in the eluate and non-bound fraction were quantified by Western Blot analysis, RIPA buffer yielded the greatest TOP2A recovery in the eluate. However, there was still substantial TOP2A remaining in the non-bound fraction (See FIG. 7, Lanes 1 and 2). In contrast, cells lysed with Cell Membrane Lysis Buffer followed by Nuclear Membrane Lysis Buffer displayed the greatest TOP2A depletion as evident from the least amount of TOP2A remaining in the non-bound fraction (Lane 6) and substantial TOP2A recovery in the eluate (See FIG. 7, Lane 5). Cells lysed with CHAPS Lysis Buffer displayed the weakest TOP2A signal (See FIG. 7, Lane 3). Resultantly, the combination of Cell Membrane Lysis Buffer followed by Nuclear Membrane Lysis Buffer was chosen as the lysis procedure.

Testing Effects of Input Cell Number, Amount of Antibody for IP and Protein G Magnetic Bead Incubation Time on TOP2A Immunodepletion from Sonicated Lysate

Since the experiment corresponding to FIG. 7 started with 50×10⁶ cells in each sample and complete depletion did not occur, the next experiment tested two different quantities of cells to determine if using less cells resulted in better depletion. Western blot analysis was utilized to determine the effects of starting cell number (10×10⁶ CEM cells v. 30×10⁶ CEM cells) as well as the effects of the quantity of primary α-TOP2A rabbit IgG (Kamiya) (5 μg v. 10 μg) and Protein G magnetic bead incubation time (10 minute v. 30 minute v. overnight) on TOP2A recovery and immunodepletion from the sonicated nuclear lysate. Results show that 5 μg of α-TOP2A rabbit IgG was insufficient to deplete TOP2A from sonicated nuclear lysates from 30×10⁶ cells at any incubation time. This is evident because the non-bound fractions contain a large amount of TOP2A and there is little TOP2A in the eluates. When fewer cells (10×10⁶) and double the amount of antibody (10 μg of α-TOP2A rabbit IgG) were used, either 10 minute or 30 minute Protein G magnetic bead incubation times resulted in more TOP2A recovery in the eluate compared to overnight incubation. Ten×10⁶ cells and a 10 minute Protein G magnetic bead incubation time were used as conditions going forward. Since there still remained a large amount of TOP2A in the non-bound fractions, the sonicated nuclear lysates were not being depleted of TOP2A. Depletion of TOP2A from the sonicated nuclear lysate is desirable in certain assays in order to quantify differences in TOP2A-bound DNA between samples.

Testing Effect of Freezing after Sonication and of Repeating the IP on Achievement of Immunodepletion of TOP2A from the Sonicated Lysate

In order to determine if additional TOP2A could be recovered from the non-bound fraction, a second round of IP was done. However, before this second round, another variable was introduced. This variable was freezing the non-bound fraction that was to be subjected to repeat IP, before further processing. It was noticed that TOP2A depletion improved by freezing, presumably because of resultant protein denaturation. This will be described below.

Following overnight freezing of the non-bound fraction, 10 μg of fresh α-TOP2A rabbit IgG and then 50 μL of fresh Protein G magnetic beads were added. Western blot analysis was used to quantify the amount of TOP2A in the non-bound fraction from the initial round of IP compared to the second round of IP. Results show that the non-bound fraction from the second round of IP was 95% depleted of TOP2A, confirmed by the amount of TOP2A in the eluate after the second round (beads were not pooled from the two IP rounds).

The results from the Western blot led to the thought that freezing the non-bound fraction overnight had increased TOP2A depletion from the non-bound fraction. To determine if this was the case, or whether the additional antibody and beads were responsible for increased depletion, the next experiment tested whether using double the amount of α-TOP2A rabbit IgG (10 μg v. 20 μg) in a single IP increased TOP2A depletion. Western blot analysis showed no significant change in TOP2A depletion from the sonicated nuclear lysate with 10 μg compared to 20 μg of α-TOP2A IgG. This is evident from the amount of TOP2A remaining in the non-bound fractions. Therefore, doubling the amount of antibody failed to provide complete TOP2A depletion from the non-bound fraction after a single IP.

Since overnight freezing had an unanticipated favorable effect on achievement of depletion of TOP2A from the sonicated nuclear lysate in the second round of IP on the following day, it was thought that the first round of IP prior to freezing may not be necessary. Therefore, an experiment was done in which the sonicated nuclear lysate was stored overnight at −20° C. before IP. The following day, a single round of IP was performed using 10 μg of α-TOP2A mouse IgG₁ and 50 μL of Protein G magnetic beads. Western blot analysis confirmed that overnight storage of the sonicated nuclear lysate at −20° C. prior to IP allowed for complete depletion of TOP2A. From this point on, all sonicated lysates were frozen overnight at −20° C. before IP.

At this point two major changes were implemented. The first was that RIPA Lysis Buffer would now be used to lyse cells. The TOP2A signal produced after using RIPA Lysis Buffer was previously thought to be too strong to deplete from the sonicated lysate. However, since the progress of the experiments thus far had shown that increased depletion is possible, it was decided to attempt using this lysis procedure to maximize the TOP2A signal. The second change was that an antibody to the non-nuclear protein, BECN1, was selected to be tested as an appropriate negative control antibody for the IP.

The new conditions were applied of using RIPA Lysis Buffer along with either α-TOP2A rabbit IgG or the negative control antibody, α-BECN1 (H-300) rabbit IgG. Western blot analysis showed that when this non-nuclear IP negative control was used, TOP2A was appropriately retained in the non-bound fraction. Therefore, α-BECN1 (H-300) rabbit IgG was used as the negative control antibody for IP going forward.

As expected, now that RIPA Lysis Buffer was being utilized to lyse cells, a large amount of TOP2A remained in the non-bound fraction that corresponded to the sonicated lysate in which α-TOP2A rabbit IgG was added, even after overnight freezing of the lysate. In order to further deplete TOP2A, two additional rounds of fresh 10 μg of α-TOP2A rabbit IgG and 50 mL Protein G magnetic beads were added to the non-bound fraction. Western blot analysis showed that after three rounds of IP, TOP2A was completely depleted from the non-bound fraction.

Analysis of Input DNA and DNA Released from TOP2A-DNA Complexes

### Demonstration of Proper Sonication in Input DNA (Sonicated Lysate)

Electrophoresis of input DNA on a 2% agarose gel contained fragments ranging from about 500 bp to about 100 bp. This demonstrates that sonication properly fragmented DNA to segments of approximately 500 bp or smaller. Obtaining fragments of this size is desirable for use in Q-PCR and high-throughput sequencing.

Q-PCR Analysis of Input DNA and DNA Released from TOP2A-DNA Complexes

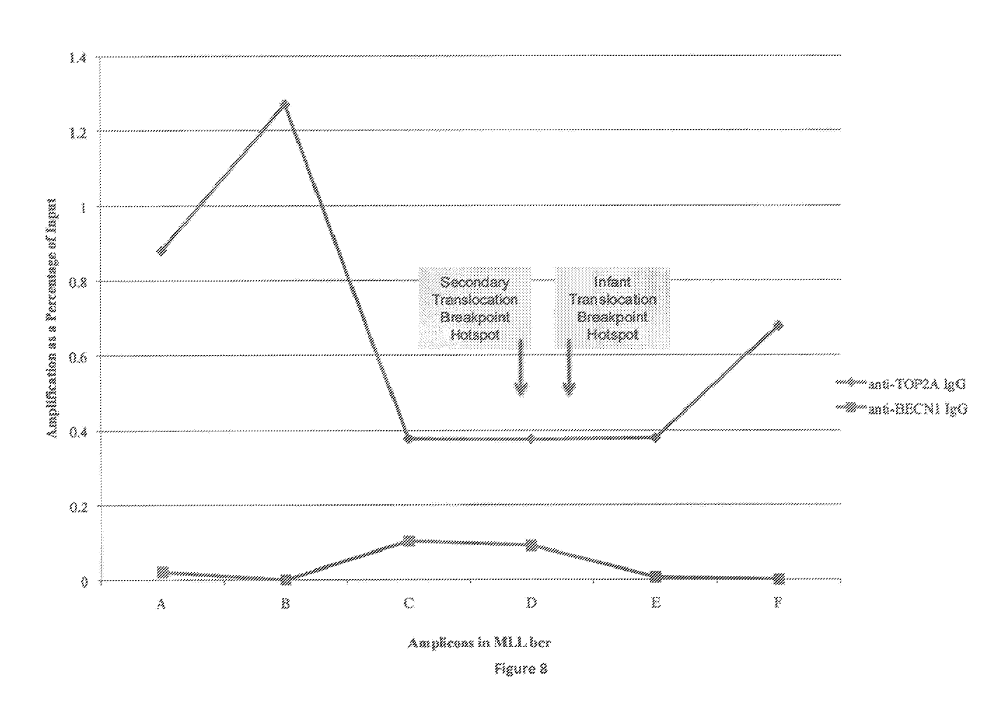

Purified input DNA and purified DNA released from TOP2A-DNA cleavage complexes after three rounds of IP (with 10 μg of either α-TOP2A rabbit IgG or α-BECN1 IgG negative control) and CIP treatment of the combined bound fraction were analyzed using Q-PCR to quantify enrichment of TOP2A-bound MLL as a percentage of that in input compared to the α-BECN1 IgG negative IP control (See FIG. 4, Steps 4a and 9a). Q-PCR results show significant enrichment of amplicons A-F, which were designed 3′ within intron 8 (intron 11 in Nilson numbering system (Nilson et al. (1996) Br. J. Haematol., 93:966-972)) of the MLL bcr because of previous evidence that MLL translocation breakpoints in leukemia occur with biased hotspots in this region (Gillert et al. (1999) Oncogene, 18:4663-4671; Megonigal et al. (2000) Proc. Natl. Acad. Sci., 97:2814-2819; Langer et al. (2003) Genes Chromosomes Cancer, 36:393-401; Megonigal et al. (1997) Proc. Natl. Acad. Sci., 94:11583-11588; Domer et al. (1995) Leukemia, 9:1305-1312; Leis et al. (1998) Leukemia, 12:758-763; Raffini et al. (2002) Proc. Natl. Acad. Sci., 99:4568-4573). Amplicon B showed the highest amplification (more than ten-fold over negative IP control using α-BECN1 IgG), followed by amplicons A and F (See FIG. 8). Note that primer pair A is in a repeat region and amplification of amplicon A could be the result of other topoisomerase II binding spots in repeat elements. Although RepeatMasker analysis showed that amplicons B and F were in repeats, further testing using BLAT and BLAST (see Methods) led to the conclusion that these amplicons were sufficiently unique for Q-PCR analysis, and that they represent the intended region in the MLL bcr. Notably, amplicons B and F are near, but not within, translocation breakpoint hotspots. It is hypothesized that amplicons C, D and E show lower enrichment than amplicons B and F compared to the α-BECN1 IgG negative IP control because of their close proximity to breakpoint hotspots, since the polymerase in the Q-PCR cannot amplify across a broken region of DNA due to TOP2A cleavage. However, since B and F are a short distance from hotspots, amplification is much higher since the polymerase can extend the full amplicon.

Amplicons that spanned other regions of the MLL bcr (amplicons G-K) did not show significant enrichment. These amplicons were not designed around regions of the MLL bcr that were shown to be biased hotspots of translocation breakpoints. This indicates that the native TOP2A cleavage complexes (without any topoisomerase II poison) near these amplicons may be too short lived to detect with this assay. Even though MLL translocation breakpoints have been identified by molecular cloning in introns throughout the entire MLL bcr, and these translocation breakpoints are near amplicons G-K, it also is important to note here that the TOP2A cleavage complexes that were studied so far were only those obtained without any drug treatment, i.e. native cleavage complexes. The control amplicons designed in MLL exon 23 and MYC, also did not show significant amplification, as expected.

### Example 2

As stated herein above, many cytotoxic anticancer drugs, at least one and often several of which are mainstays of virtually all anticancer chemotherapy, are “TOP2 poisons.” Examples include, without limitation, epipodophyllotoxins, anthracyclines, the anthracenedione mitoxantrone, and dactinomycin. TOP2 poisons convert native TOP2 into a cellular toxin by disrupting the cleavage re-ligation equilibrium, either by decreasing the reverse rate of re-ligation or increasing the forward rate of cleavage, both of which increase cleavage complexes and cause DNA strand breaks, which can initiate apoptosis or promote illegitimate DNA recombination. Not only are these agents cytotoxic, they are associated with secondary leukemia as a significant, deadly chemotherapy complication. Associations of chemotherapeutic TOP2 poisons with treatment-related secondary leukemias have implicated TOP2 in the DNA damage that leads to translocations. Furthermore, MLL translocations in infant leukemia originate in utero, population epidemiology studies have indicated that maternal-fetal exposures to dietary TOP2 interacting substances increase the risk of infant AML, and these agents are known to cross the placenta. Also, molecular epidemiology linked an inactivating genetic variant of NQ01, which detoxifies the benzene metabolite and TOP2 poison p-benzoquinone found in cigarette and wood smoke, with MLL translocations, particularly the t(4;11), the most common MLL translocation in infant ALL. These observations favor a model where direct repair of topoisomerase II (TOP2) mediated damage, which can be induced by anticancer drugs as well as dietary substances and environmental toxins, creates translocations.

A body of additional evidence also favoring this model derives from biochemical in vitro cleavage assays demonstrating that MLL translocation breakpoints are functional TOP2 cleavage sites that could be resolved to form the breakpoint junctions observed in leukemias in patients, and that cleavage at these sites is enhanced by chemotherapeutic, dietary or environmental TOP2 poisons. Additionally, the translocation breakpoint hotspot in the PML gene in secondary acute promyelocytic leukemia (APL) with the t(15;17) in patients previously treated with mitoxantrone (e.g., for primary breast cancer or multiple sclerosis, for which mitoxantrone has been administered to patients in the clinic) is a preferred site of formation of mitoxantrone-stimulated TOP2 cleavage complexes, establishing a cause-and-effect relationship between mitoxantrone induced TOP2 cleavage and treatment-related secondary APL.

Expression of TOP2A, which is essential for DNA replication, is cell cycle dependent and down-regulated in quiescent cells. The beta isoform of TOP2 (TOP2B) is important during transcription, particularly in activation or repression of genes that are regulated during development (Nitiss, J. L. (2009) Nat. Rev. Cancer, 9:338-350; Nitiss, J. L. (2009) Nat. Rev. Cancer, 9:327-337; Lyu et al. (2006) Mol. Cell. Biol., 26:7929-7941). Studies on TOP2B are of interest because primitive hematopoietic progenitor and stem cells such as those in umbilical cord blood, which are used as described below to model TOP2 DNA damage in cells that more closely mimic target cells of MLL gene translocations, are generally quiescent (Srour et al. (2002) Methods Mol. Med., 63:93-111). Furthermore, a non-hematopoietic cell murine model suggests a role of proteolytic processing at Top2B cleavage complexes in etoposide induced recombination (Azarova et al. (2007) Proc. Natl. Acad. Sci., 104:11014-11019). The proteosomal degradation of genistein-induced Top2B cleavage complexes may also expose DNA double strand breaks and lead to rearrangements, which may lead to leukemia in infants (Azarova et al. (2010) Biochem. Biophys. Res. Commun., 399:66-71). Therefore, the novel assays of the instant invention were performed to immunodepelete TOP2B for the same purpose.

In this assay, by taking advantage of the covalent phosphodiester bonds between TOP2A or TOP2B and DNA, the activity of calf intestinal phosphatase (CIP) (i.e. hydrolysis of phosphodiester bonds via removal of 5′ phosphates) is used to release DNA from cleavage complexes at exact sites of cleavage. The assay comprised the steps of:

1. Treating cells with topoisomerase II poison or proceeding with untreated cells to study native TOP2A or TOP2B cleavage;

2. Lysing cell and nuclear membranes using a lysis buffer (e.g. RIPA);

3. Sonicating lysate to fragment DNA into ˜500 bpy segments;

4. Reserving 10% of sonicated lysate from Step 3. The reserved lysate is to be used for Q-PCR analysis of enrichment of TOP2-bound MLL after successive purification as % of that in 5% of sonicated lysate, as input (See Step 9a), and for quantification of TOP2 in 5% of sonicated lysate, as input, by Western Blot analysis (See Step 11);

5. Immunoprecipitating TOP2A or TOP2B including DNA-bound TOP2A or TOP2B including DNA-bound TOP2A or TOP2B. The immunoprecipitation is performed by adding α-TOP2A or α-TOP2B IgG to sonicated lysate to bind TOP2A or TOP2B (including DNA-bound TOP2A or TOP2B); binding α-TOP2A or α-TOP2B IgG to Protein G magnetic beads; and using a magnet to separate Protein G magnetic bead-bound fraction from non-bound fraction. The immunoprecipitation may be repeated on the non-bound fraction (e.g., at least two more times) using fresh α-TOP2A or α-TOP2B IgG and Protein G magnetic beads.

6. After each round of immunoprecipitation, saving 5% of non-bound fraction (non-bound TOP2 including DNA-bound TOP2 from sonicated lysate) to quantify non-bound TOP2 in 5% of non-bound fraction by Western blot analysis for evidence of depletion from sonicated lysate;

7. Combining bound fractions from the (3) rounds of immunoprecipitation;

8. Treating combined bound fraction with calf intestinal phosphatase (CIP) to release TOP2-bound DNA from TOP2 cleavage complexes;

9. Analyzing 10% of DNA released from DNA-TOP2 cleavage complexes in bound fraction (See Step 8) by Q-PCR analysis to quantify enrichment of TOP2-bound MLL (or other desired target sequence) after successive purification by IP (Step 5) and release (Step 8) as a % of that in input (Steps 3, 4a);

10. Heating the bound fraction after DNA release (See Step 8) at 70° C. to elute TOP2 from Magnetic Beads;

11. Quantifying TOP2 in input, 5% of non-bound fractions, and final eluate by Western blot analysis;

12. Pooling DNA released from DNA-TOP2 cleavage complexes from three individual rounds of steps 1-8 (after removal of 10% for Q-PCR) for high-throughput sequencing to map and quantify DNA ends created by TOP2 cleavage genome-wide

These steps were performed in CEM cells and in fresh cord blood mononuclear cells (MNCs) to better mimic target cells for translocations. Western blot and Q-PCR analyses proved that the following was achieved: 1) isolation and immunodepletion of TOP2B and TOP2B-bound DNA, 2) CIP release of TOP2-bound DNA from the cleavage complexes formed in fresh cord blood MNCs, and 3) quantitative enrichment of DNA amplicons near known MLL translocation breakpoint hotspots using α-TOP2A antibody for immunodepletion over that obtained using a negative control antibody for immunodepletion in fresh cord blood MNCs. This allows for the localization of cleavage complexes at single base resolution genome-wide through high-throughput sequencing of DNA ends created by TOP2 and mapping them to the genome.

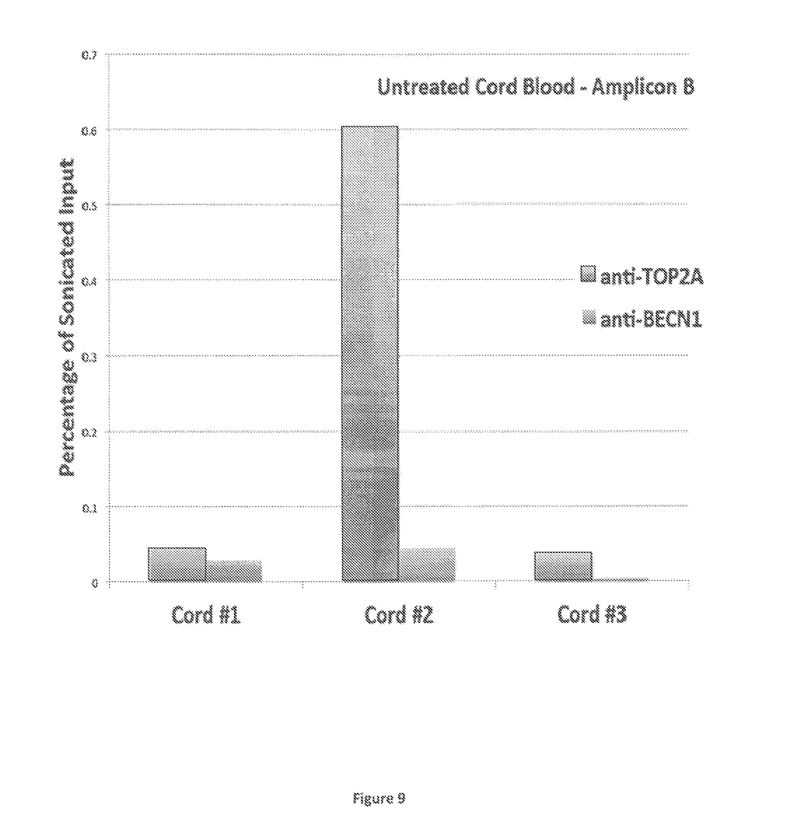

FIG. 9 shows the Q-PCR analysis of DNA released by CIP treatment showing quantitative enrichment of DNA amplicon (amplicon B; positions 6226-6294 relative to Reference sequence GenBank No. U04737) proximal to the MLL translocation breakpoint hotspot in bound fractions obtained using α-TOP2A antibody for immunodepletion over that obtained using negative control antibody α-BECN1 for immunodepletion in mononuclear cells from three untreated cord blood samples.

Three successive rounds of immunoprecipitation were performed using either α-TOP2A or α-BECN1 (negative IP control) and incubation with Protein G magnetic beads. DNA was released from cleavage complexes by CIP treatment. Amplification was plotted as a percentage of that in input (5% of sonicated lysate). DNA prepared in this fashion can then be analyzed by high throughput sequencing.

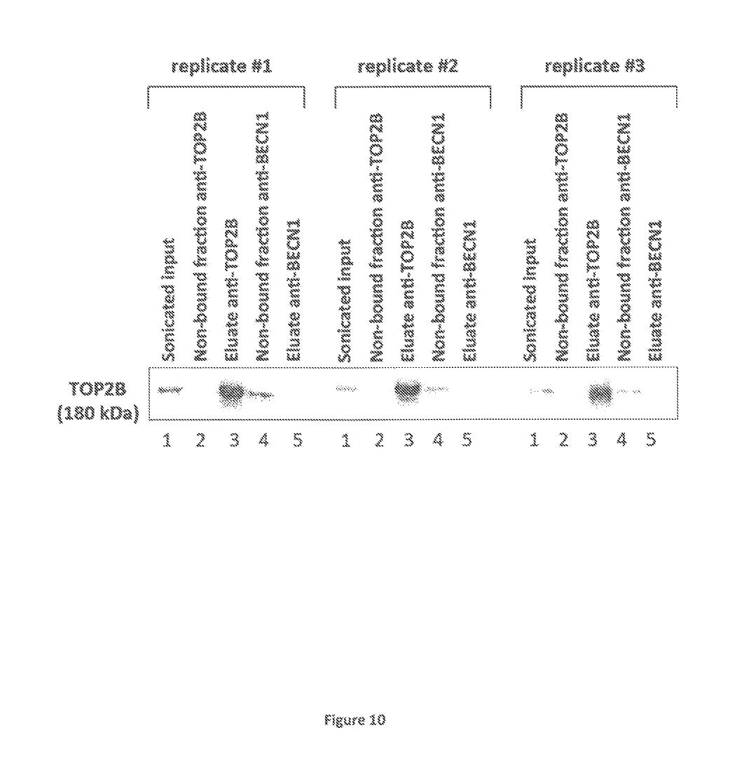

FIG. 10 shows the immunodepletion of TOP2B from untreated CEM cells. CEM cells (10×10⁶) were lysed with RIPA Buffer. Lysates were passed through 25-G and 27-G needles and stored at −20° C. overnight, sonicated, and each replicate separated into equal halves, immunoprecipitation of which was performed with 10 μg rabbit α-TOP2B IgG (Santa Cruz) or, as a negative control antibody, 10 μg rabbit α-BECN1 H-300 IgG (Santa Cruz, Calif.)×1.5 h at 4° C. The bound fraction was treated with calf intestinal phosphatase (CIP) at 200 units/mL final concentration for 1 hour at 37° C. to release TOP2-bound DNA from TOP2 cleavage complexes. Bound TOP2B or BECN1 was removed by incubation at room temperature×10 minutes with Protein G magnetic beads (Millipore). Immunoprecipitated fractions, 5% of sonicated inputs and 5% of non-bound fractions, were analyzed by Western blot using α-TOP2B (H-8) mouse monoclonal IgG₁ (Santa Cruz) and Amersham ECL Plus Western blotting detection system (GE Healthcare). Note full depletion of TOP2B in halves precipitated with α-TOP2B, and no depletion in halves precipitated with α-BECN1. Similar to the TOP2A complexes the TOP2B complexes in CEM cells can then be sequenced.

### Example 3

A conventional in vitro topoisomerase II cleavage assay has been used in order to quantify and determine the location of cleavage complexes in naked, 5′ end labeled, double stranded DNA substrates (plasmid subclones or oligonucleotides). Briefly, in this conventional assay the substrate is prepared by treatment of the plasmid with DNA ligase to remove any nicks, excision of the desired substrate fragment from the plasmid by restriction enzyme cleavage, followed by dephosphorylation using CIP and then 5′ radiolabeling of both 5′ ends in a kinase reaction using [γ³²P]ATP, and further restriction enzyme cleavage to generate a substrate that is labeled at only one 5′ end. After purification, the double stranded substrate with one 5′ end labeled is treated with recombinant topoisomerase II in the presence of ATP with or without topoisomerase II poisons, followed by trapping of the cleavage complexes with SDS, addition of EDTA to stop the cleavage reaction, treatment with proteinase K to deproteinize the cleavage complexes and ethanol precipitation of the singly 5′ end labeled cleaved double stranded substrate. The cleaved double stranded substrate is then heat denatured to make it single stranded, and denaturing polyacrylamide gel electrophoresis of the cleavage assay reaction products with a DNA sequencing ladder primed at the same 5′ end run in parallel, is employed in order to map the TOP2 cleavage sites as determined by migration of the cleaved radiolabeled fragments on the gel (Kolaris et al. (2005) ASH Annual Meeting Abstracts, 106:2850; Felix et al. (2006) DNA Repair (Amst), 5:1093-1108; Lovett et al. (2001) Proc. Natl. Acad. Sci., 98:9802-9807; Whitmarsh et al. (2003) Oncogene, 22:8448-8459; Robinson et al. (2008) Blood 111:3802-3812; Lovett et al. (2001) Biochem., 40:1159-1170; Mistry et al. (2005) N. Engl. J. Med., 352:1529-1538; Hasan et al. (2008) Blood 112:3383-90; Lindsey et al. (2004) Biochem., 43:7563-7574). In these conventional in vitro cleavage assays, native or drug stimulated topoisomerase II cleavage complexes can be mapped at single base precision within the sequence of inquiry, and correlations have been identified between cleavage sites and translocation breakpoints. However, the assay has a number of significant limitations including that it is tedious, not high throughput, requires radiolabeling and, furthermore, the substrates are limited to the size of the DNA sequencing ladder run in parallel with the reaction products of the cleavage assay (generally only a few hundred bases) that can be resolved by electrophoresis on a polyacrylamide gel. Furthermore, analysis of cleavage assay reaction products on a denaturing gel yields information on the sites of cleavage by TOP2 on a single strand of DNA in isolation and will not give information as to whether bona fide DNA double strand breaks have occurred unless both strands are examined separately. Alternatively, the reaction products can be examined by non denaturing polyacrylamide gel electrophoresis; however, the latter will only inform the approximate sizes of the cleaved fragments without exact base precision because denaturing polyacrylamide gel electrophoresis is required the sequencing.

Determination of whether bona fide double strand breaks have occurred is important because double occupancy by drug at both scissile bonds of a topoisomerase II cleavage site is needed for double strand cleavage with etoposide, which has implications that anticancer agents and other TOP2 poisons may stimulate topoisomerase II single stranded nicks in DNA rather than double stranded breaks (Bromberg et al. (2003) J. Biol. Chem., 278:7406-7412). Alternatively, single strand nicks are kinetic intermediates of topoisomerase II DNA double strand breaks, and there may be more single strand nicks vs. double strand breaks at a subset of cleavage sites at any given time (Lovett et al. (2001) Proc. Natl. Acad. Sci., 98:9802-9807).

The methods of the instant invention can be used to improve the above conventional methods. The methods employ the release of DNA from TOP2 cleavage complexes by hydrolysis of phosphodiester bonds (e.g., with CIP) and high-throughput sequencing in order to map TOP2 cleavage sites in vitro with exact base precision in a more expeditious and high throughput manner over larger sequence regions and in both strands of the substrate. The methods may comprise the following steps:

1. Preparing plasmid subclone containing entire 8.3 kb DNA fragment spanning MLL bcr or other desired substrate. The insert size is non-limiting.

2. Optionally treating the double stranded DNA plasmid substrate with T4 DNA ligase to assure that substrate is not nicked. Plasmids may be nicked due to freeze-thawing.

3. Releasing insert (substrate) for analysis from plasmid (e.g., by restriction enzyme cleavage).

4. Treating with CIP to prevent re-ligation of the substrate after restriction enzyme cleavage and heat inactivating the CIP.

5. Purifying the released double-stranded substrate (e.g., on a gel).

6. Subjecting the purified double stranded substrate to in vitro cleavage in the presence/absence of a TOP2 poison in reaction mixtures containing recombinant TOP2 (e.g., TOP2A or TOP2B), ATP, divalent cation (Mg²⁺).

7. Transferring reaction products to a buffer (e.g., cell lysis buffer) and then proceeding with same steps as in cell based assay (see below). The lysate may, optionally, be sonicated to fragment DNA into ˜500 bp segments.

8. Reserving 10% of cleaved DNA in lysis buffer from Step 7 for Q-PCR analysis of enrichment of TOP2-bound MLL after successive purification as % of that in 5% of sonicated cleaved DNA in lysis buffer, as input (See Step 13) and for quantification of TOP2 in 5% of sonicated lysate, as input, by Western Blot analysis (See Step 15).

9. Immunoprecipitating TOP2 including DNA-bound TOP2. The immunoprecipitation may comprise adding α-TOP2 IgG to cleaved substrate in lysis buffer to bind TOP2 (including DNA-bound TOP2), binding α-TOP2 IgG to Protein G magnetic beads; and using magnet to separate Protein G magnetic bead-bound fraction (TOP2 including DNA-bound TOP2) from non-bound fraction. These steps may be repeated on non-bound fraction more than once (e.g., two more times) using fresh α-TOP2 IgG and Protein G magnetic beads.

10. Saving 5% of non-bound fraction (non-bound TOP2 including DNA-bound TOP2 from sonicated cleaved substrate) to quantify non-bound TOP2 in 5% of non-bound fraction by Western blot analysis for evidence of depletion from cleaved substrate in lysis buffer.

11. Combining bound fractions from all rounds of Step 9.

12. Treating combined bound fraction with calf intestinal phosphatase (CIP) to release TOP2-bound DNA from TOP2 cleavage complexes.

13. Analyzing 10% of DNA released from DNA-TOP2 cleavage complexes in bound fraction (See Step 11) by Q-PCR analysis to quantify enrichment of TOP2-bound MLL after successive purification by IP (Step 9) and release (Step 12) as a of that in input (Step 8).

14. Heating the bound fraction after DNA release (See Step 12) at 70° C. to elute TOP2 from magnetic beads.

15. Quantifying TOP2 in input, 5% of non-bound fractions, and final eluate by Western blot analysis.

16. Pooling DNA released from DNA-TOP2 cleavage complexes from three individual rounds of steps 1-12 (after removal of 10% for Q-PCR) for high-throughput sequencing to map and quantify DNA ends created by TOP2 cleavage genome-wide.

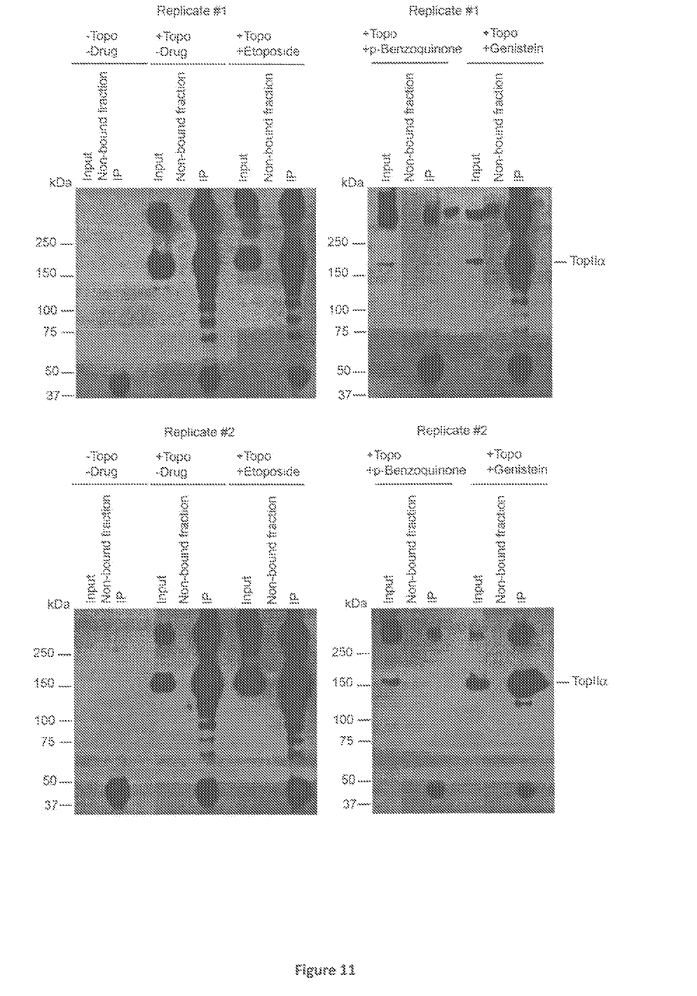

The above nonradioactive pull-down assay with release of DNA from cleavage complexes by CIP was performed using an 8.3 kb double stranded fragment of the MLL bcr as substrate. FIG. 11 shows the immunodepletion of TOP2A following in vitro cleavage by the native enzyme or by TOP2A in the presence of the TOP2 poisons etoposide, genistein, or p-benzoquinone. Specifically, 0.5 μg of substrate DNA per reaction was subjected to in vitro cleavage by 2 μg recombinant human TOP2A alone or in the presence of the TOP2 poisons etoposide, genistein or p-bezoquinone at 20 μM final concentration. The reaction products in which cleavage-religation equilibria were established for 10 minutes at 37° C., were immediately transferred directly into RIPA Buffer Immunoprecipitation was performed twice with 10 μg rabbit polyclonal α-TOP2A IgG (Kamiya; Seattle, Wash.) for 1 hour at 4° C. Bound TOP2A was removed by incubation at room temperature for 10 minutes with Protein G magnetic beads (Millipore; Billerica, Mass.). The bound fraction on magnetic beads was treated with calf intestinal phosphatase (CIP) at 200 units/mL final concentration for 1 hour at 37° C. to release TOP2A-bound DNA from TOP2A cleavage complexes Immunoprecipitated fractions, 5% of inputs and 5% of non-bound fractions, were analyzed by Western blot using α-TOP2A mouse monoclonal IgG₁(Santa Cruz; Santa Cruz Calif.) and Amersham ECL Plus Western blotting detection system (GE Healthcare). Note full depletion of TOP2A following IP with α-TOP2A. Also note higher MW species consistent with TOP2A homodimers or, alternatively, gel shift resulting from residual DNA bound TOP2A following the CIP treatment.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for identifying sequences present in protein-nucleic acid complexes, comprising:
a) cleaving said protein-nucleic acid complexes by contacting the protein-nucleic acid complexes with a phosphatase;
b) contacting the free nucleic acid molecules with a polymerase and a polynucleotide kinase,
c) ligating adaptors to the free nucleic acid molecules;
d) amplifying the free nucleic acid molecules of step c); and
e) identifying the sequence of the amplified nucleic acid molecules, thereby identifying the sequences present in said protein-nucleic acid complexes.

2. The method of claim 1, wherein said protein-nucleic acid complex is in a cell.

3. The method of claim 2, further comprising isolating said protein-nucleic acid complexes from the cell prior to step a).

4. The method of claim 1 performed in vitro.

5. The method of claim 1, wherein the nucleic acid in said protein-nucleic acid complexes is genomic DNA.

6. The method of claim 1, wherein the identification of the sequences in step e) comprises sequencing the amplified nucleic acid molecules from step d).

7. The method of claim 2, wherein said cell has been exposed to an agent suspected of modulating formation of protein-nucleic acid complexes.

8. The method of claim 7, wherein said cell has been exposed to a topoisomerase II poison.

9. The method of claim 2, wherein said cells are obtained from a subject.

10. The method of claim 9, wherein said subject has been exposed to an agent suspected of modulating formation of protein-nucleic acid complexes.

11. The method of claim 10, wherein said subject has been exposed to a topoisomerase II poison.

12. The method of claim 8, wherein said topoisomerase II poison is selected from the group consisting of anthracyclines, doxorubicin, idarubicin, daunorubicin, epipodophyllotoxins, etoposide, etoposide metabolite, etoposide quinone, etoposide catechol, teniposide, aminoacridines, amsacrine, anthracenediones, mitoxantrone, actinomycines, dactinomycin, benzene, benzene metabolite, benzoquinone, 1,4-benzoquinone, m-AMSA, NK314, XK469, dietary TOP2 interacting substances, genistein, quercitin, catechin, bioflavinoid, environmental factor, pollutant, and a pesticide.

13. The method of claim 3, wherein said isolating of protein-nucleic acid complexes from said cell comprises lysing said cells and immunoprecipitating said protein-nucleic acid complexes.

14. The method of claim 1, wherein the nucleic acid of the protein-nucleic acid complexes has been fragmented prior to step a).

15. The method of claim 1, wherein the protein of the protein-nucleic acid complex is selected from the group consisting of topoisomerases, methylases, glycosylases, and RNA enzymes.

16. The method of claim 15, wherein the protein of the protein-nucleic acid complexes is topoisomerase II, TOP2A, TOP2B, or other topoisomerase related molecule.

17. The method of claim 1, wherein the free nucleic acid molecules are isolated prior to step b).

18. The method of claim 1, wherein said polymerase of step b) is Klenow.

19. The method of claim 1, wherein the polynucleotide kinase of step b) is T4 polynucleotide kinase.

20. The method of claim 1, wherein step c) comprises adding at least one 3′ overhang nucleotide to the free nucleic acid molecules of step b) prior to contacting with the adaptors.

21. The method of claim 1, wherein step d) comprises amplifying the nucleic acid molecules with primers specific to the adaptors.

22. The method of claim 1, wherein the protein-nucleic acid complex of step a) is obtained by cloning a target sequence into a plasmid, optionally treating the double stranded DNA plasmid with T4 DNA ligase, releasing the substrate from the plasmid, treating the released substrate with a phosphatase, inactivating the phosphatase, purifying the substrate, and contacting the purified substrate with said protein, optionally in the presence of an agent suspected of modulating formation of protein-nucleic acid complexes.

23. A kit for performing the method of claim 1.

24. The kit of claim 23, comprising:
a) a solid support and a buffer for isolating protein-nucleic acid complexes;
b) a polymerase;
c) a polynucleotide kinase; and
d) a phosphatase.

25. The kit of claim 23, further comprising at least one of
a) an agent or a buffer for lysing cells;
b) at least one adaptor;
c) at least one primer specific for said adaptor;
d) antibody immunologically specific for the antibody of the protein of said protein-nucleic acid complex; and
e) instruction material.

26. The kit of claim 23, wherein said phosphatase is calf intestinal phosphatase.

27. The kit of claim 23, wherein said polynucleotide kinase is T4 polynucleotide kinase.

28. The kit of claim 23, wherein said polymerase is Klenow.

29. The kit of claim 25, wherein said antibody is immunologically specific for topoisomerase II.


SEQUENCE LISTING (26 sequences)

SEQ ID NO: 1 - DNA, 22 bp, Artificial Sequence
  Primer
cacgcctataatcccagcactt

SEQ ID NO: 2 - DNA, 21 bp, Artificial Sequence
  Primer
gcctgtgatcccagctactca

SEQ ID NO: 3 - DNA, 30 bp, Artificial Sequence
  Primer
ccagaccttacaactgtttcgtatattaca

SEQ ID NO: 4 - DNA, 30 bp, Artificial Sequence
  Primer
ttctatttccactggtattaccactttagt

SEQ ID NO: 5 - DNA, 21 bp, Artificial Sequence
  Primer
tgagcccttccacaagttttg

SEQ ID NO: 6 - DNA, 22 bp, Artificial Sequence
  Primer
tatccctggactcaaccaacct

SEQ ID NO: 7 - DNA, 27 bp, Artificial Sequence
  Primer
gtatccctgtaaaacaaaaaccaaaag

SEQ ID NO: 8 - DNA, 24 bp, Artificial Sequence
  Primer
gtgaaggcaaatagggtgtgattt

SEQ ID NO: 9 - DNA, 24 bp, Artificial Sequence
  Primer
ctaagcaaaaaattccagcagatg

SEQ ID NO: 10 - DNA, 28 bp, Artificial Sequence
  Primer
ccatccaaagttgtgtaattgtaaaact

SEQ ID NO: 11 - DNA, 22 bp, Artificial Sequence
  Primer
cagggtggtttgctttctctgt

SEQ ID NO: 12 - DNA, 20 bp, Artificial Sequence
  Primer
gccgtcctccagtcgtagag

SEQ ID NO: 13 - DNA, 20 bp, Artificial Sequence
  Primer
tcggacaccgaggaggaatg

SEQ ID NO: 14 - DNA, 23 bp, Artificial Sequence
  Primer
gttagcgaggatggtctcaatct

SEQ ID NO: 15 - DNA, 17 bp, Artificial Sequence
  Primer
gcaaccctccgcctgtt

SEQ ID NO: 16 - DNA, 28 bp, Artificial Sequence
  Primer
aaacaaagagctatgggaatataaagga

SEQ ID NO: 17 - DNA, 30 bp, Artificial Sequence
  Primer
agtagtccactggcatatatttaattcaat

SEQ ID NO: 18 - DNA, 21 bp, Artificial Sequence
  Primer
cgacgacaacaccaattttcc

SEQ ID NO: 19 - DNA, 28 bp, Artificial Sequence
  Primer
tgacaagaaaaagtctgttcacatagag

SEQ ID NO: 20 - DNA, 22 bp, Artificial Sequence
  Primer
ccctgagaaatggcagagaaac

SEQ ID NO: 21 - DNA, 23 bp, Artificial Sequence
  Primer
ttattgaccggaggtggtttttc

SEQ ID NO: 22 - DNA, 31 bp, Artificial Sequence
  Primer
ctttgacactcaatatactttatgatcactg

SEQ ID NO: 23 - DNA, 22 bp, Artificial Sequence
  Primer
tcccacacattttctgcttcac

SEQ ID NO: 24 - DNA, 24 bp, Artificial Sequence
  Primer
ggtacaaagaagcaggatgcctta

SEQ ID NO: 25 - DNA, 24 bp, Artificial Sequence
  Primer
caatggtcctgttttcatcatgtt

SEQ ID NO: 26 - DNA, 18 bp, Artificial Sequence
  Primer
cctctggcgctccaagac